(19) United States
(12) Patent Application Publication
(10) Pub. No.: US 2026/0253040 A1
(43) Pub. Date: Aug. 27, 2026

(54) SYSTEM

(71) Applicant: SoftBank Group Corp., Tokyo (JP)

(72) Inventor: Ryouji NISHIMOTO, Tokyo (JP)

(73) Assignee: SoftBank Group Corp., Tokyo (JP)

(21) Appl. No.: 19/536,233

(22) Filed: Feb. 11, 2026

(30) Foreign Application Priority Data
Feb. 21, 2025 (JP) 2025-027068

(51) Int. Cl. G06Q 10/1093

(52) U.S. Cl. CPC G06Q 10/1093

(57) ABSTRACT

The system according to the embodiment includes a reception unit, an analysis unit, an adjustment unit, and a notification unit. The reception unit inputs information on a medicine to be taken by a user. The analysis unit analyzes a combination or timing of the medicine based on the information input by the reception unit. The adjustment unit adjusts a timing of taking the medicine based on information analyzed by the analysis unit. The notification unit performs notification of the timing of taking the medicine based on the timing adjusted by the adjustment unit.

### CROSS-REFERENCE TO RELATED APPLICATION

The present application claims priority to and incorporates by reference the entire contents of Japanese Patent Application No. 2025-027068 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, it is difficult to appropriately manage a combination or timing of medicines, and there is a risk that a patient may take the medicine at a wrong timing.

### SUMMARY OF THE INVENTION

The system according to the embodiment includes a reception unit, an analysis unit, an adjustment unit, and a notification unit. The reception unit inputs information on a medicine to be taken by a user. The analysis unit analyzes a combination or timing of the medicine based on the information input by the reception unit. The adjustment unit adjusts a timing of taking the medicine based on information analyzed by the analysis unit. The notification unit performs notification of the timing of taking the medicine based on the timing adjusted by the adjustment unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

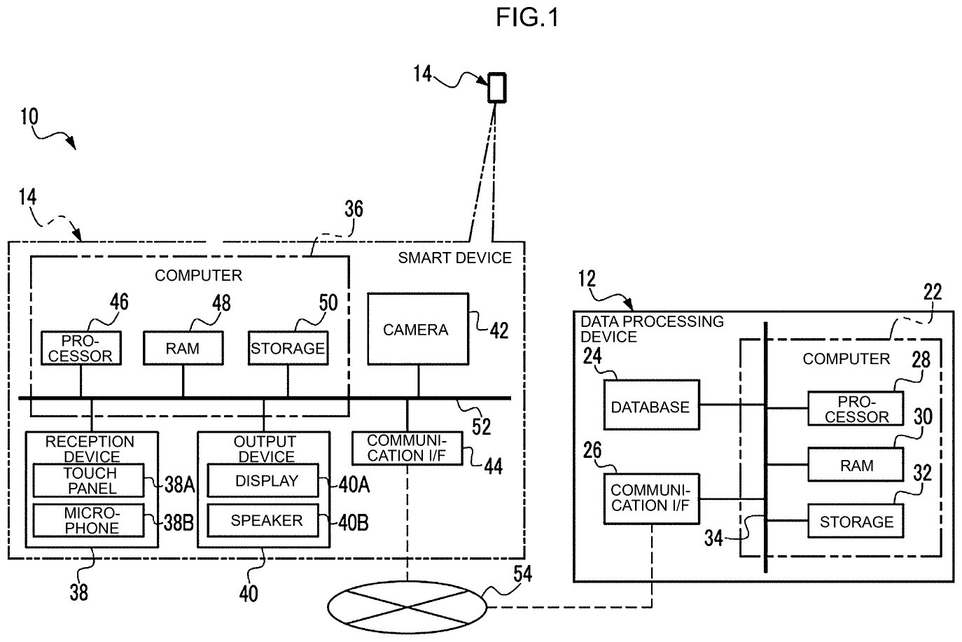

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

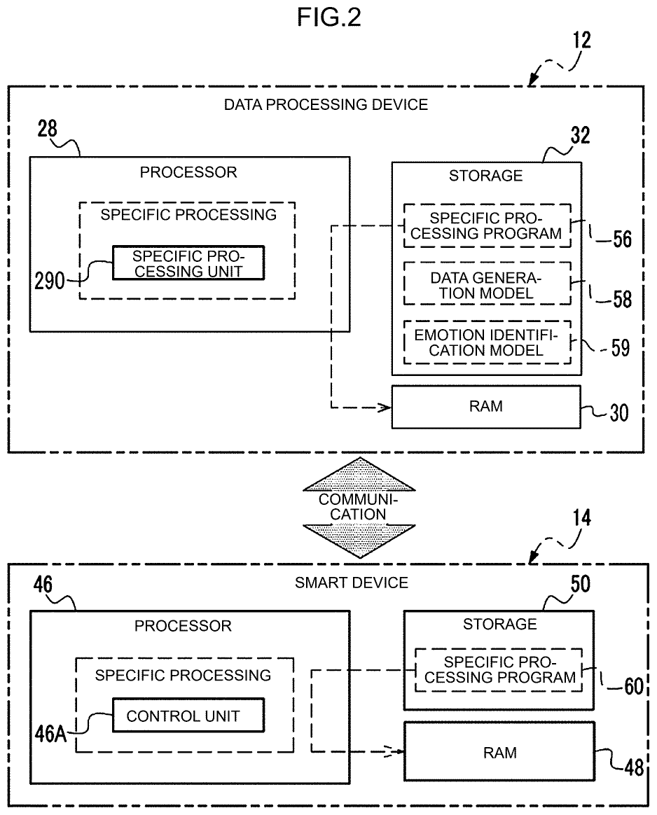

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The medicine taking schedule adjustment system according to the embodiment of the present invention is a system that inputs information on a medicine to be taken by a user, analyzes a combination, a timing, a side effect, and the like of the medicine, adjusts a timing of taking the medicine in consideration of a daily schedule of the user, and notifies the timing. This system inputs information on the medicine to be taken by the user, and includes information such as a name, a dosage, a frequency of taking, a time of taking, and a side effect of the medicine. Next, the system analyzes the combination or timing of the medicine based on the information. For example, a case where a combination of specific medicines is dangerous or a case where a side effect is reduced by taking the medicine in a specific time zone is considered. The system adjusts the timing of taking the medicine in consideration of the daily schedule of the user. For example, when there is a medicine to be taken by the user after breakfast, the system notifies the time of taking in consideration of a time of breakfast. In addition, when the user changes a schedule, the system immediately readjusts the schedule and notifies the timing of taking the medicine based on a new schedule. Furthermore, the system considers side effect information about the medicine and notifies the timing of taking by reflecting the information in a daily activity. For example, when a specific medicine causes drowsiness, the system notifies to take the medicine at night. In addition, the system integrates information on medicines prescribed at a plurality of hospitals and supports a patient having poor knowledge of medicines so that the patient can take the medicines securely. This system is a tool for comprehensively managing the combination, timing, side effect, and the like of the medicine and supporting a health of the user. For example, the user can easily manage a schedule of taking the medicine through the system and can take the medicine securely. Accordingly, the medicine taking schedule adjustment system can efficiently adjust and notify the timing of taking the medicine of the user. Specifically, the present system operates on a distributed processing infrastructure constructed on a cloud computing environment or an on-premise server, and is realized by an information processing apparatus including a processor having high computing capability and a large-capacity memory. The present system stores input medicine information as structured data in a database, and executes a simulation algorithm based on pharmacokinetics, thereby performing prediction of transition of blood concentration and quantitative evaluation of interaction risk. The present system acquires schedule information of the user as time-series data, and solves a mathematical optimization problem using this as a constraint condition, thereby calculating an optimal timing of taking that maximizes a medicinal effect while minimizing a side effect risk. For example, the present system receives high-dimensional vector data including parameters related to a half-life and a metabolic pathway of the medicine as input, and outputs a risk level of interaction as a score in a range of 0 to 1 using a multilayer neural network. When this score exceeds a predetermined threshold, the present system sets a warning flag and executes logic for searching for an alternative timing of taking. In addition, the present system monitors an action log and vital sign data of the user in real time, and triggers rescheduling processing using dynamic programming or a genetic algorithm at a moment when a schedule change or a physical condition change is detected. This makes it possible to flexibly and immediately provide an optimal schedule even for a complex and dynamic situational change that cannot be handled by static rule-based management. Furthermore, the present system uses natural language processing technology to extract unstructured text information from electronic medical record or prescription data, converts the information into a standardized medical term code, and performs integrated management, thereby ensuring data interoperability between different medical institutions. In this way, the present system performs not only mere recording and display of information but also advanced information processing fusing pharmaceutical knowledge and mathematical optimization technology, thereby producing a technical effect of dramatically improving safety and convenience for the user.

The medicine taking schedule adjustment system according to the embodiment includes a reception unit, an analysis unit, an adjustment unit, and a notification unit. The reception unit inputs information on a medicine to be taken by a user. The medicine to be taken by the user includes, for example, a medicine name, a component, a dose, a taking method, a side effect, and the like, but is not limited to such an example. The reception unit provides, for example, a text field for inputting the medicine name. In addition, the reception unit can also provide a numerical field for inputting a dosage. Furthermore, the reception unit can also provide a drop-down menu for inputting a frequency of taking or a time of taking. For example, the reception unit has a function of auto-completing a candidate medicine name when the user inputs the name of the medicine. The analysis unit analyzes a combination or timing of the medicine based on the information input by the reception unit. The analysis unit considers, for example, a case where a combination of specific medicines is dangerous or a case where a side effect is reduced by taking the medicine in a specific time zone. The analysis unit evaluates a risk of interaction based on, for example, component information of the medicine. In addition, the analysis unit can also analyze a timing of taking the medicine and propose an optimal time of taking. For example, the analysis unit collates the component information of the medicine with a database and evaluates the risk of interaction. The adjustment unit adjusts a timing of taking the medicine based on information analyzed by the analysis unit. The adjustment unit adjusts the timing of taking the medicine in consideration of, for example, a daily schedule of the user. For example, when there is a medicine to be taken by the user after breakfast, the adjustment unit notifies the time of taking in consideration of a time of breakfast. In addition, the adjustment unit can also immediately readjust the schedule when the user changes a schedule, and notify the timing of taking the medicine based on a new schedule. For example, the adjustment unit cooperates with a calendar application of the user and reflects a change in schedule in real time. The notification unit performs notification of the timing of taking the medicine based on the timing adjusted by the adjustment unit. The notification unit transmits, for example, a push notification to a smartphone of the user. In addition, the notification unit can also transmit a notification to a mail address of the user. Furthermore, the notification unit can also transmit a notification to a smart watch of the user. For example, when the user changes a schedule, the notification unit immediately readjusts the schedule and notifies the timing of taking the medicine based on a new schedule. Accordingly, the medicine taking schedule adjustment system according to the embodiment can efficiently adjust and notify the timing of taking the medicine of the user. Specifically, each unit of the present system is a module functionally realized by a hardware processor such as a CPU, a GPU, or a dedicated AI accelerator executing a program code stored in a memory. The reception unit incorporates an optical character recognition (OCR) engine or a natural language understanding (NLU) module, and has a function of automatically extracting medicine information from a prescription image captured by a camera or voice input data. The analysis unit predicts a complex interaction between medicine components at a molecular structure level using a machine learning model such as a graph neural network (GNN). Input to this analysis unit is graph data or a feature amount vector representing a chemical structure of each medicine, and output is a probability value indicating presence or absence of interaction and severity thereof. The adjustment unit includes a constraint satisfaction problem (CSP) solver or a reinforcement learning agent, defines the schedule of the user, a constraint condition of the medicine (before meal, after meal, etc.), and a side effect risk as a reward function, and generates an action plan (schedule) that maximizes a cumulative reward. The notification unit includes a messaging infrastructure that selects a communication protocol such as MQTT or HTTP/2 according to a device status or a communication environment of the user, and distributes notification data with low delay and certainty. In addition, each unit cooperates loosely via an API gateway, and by adopting a microservice architecture, independent scaling or update of each function is enabled. This improves availability and maintainability of the system as a whole, and enables stable service provision even for a large amount of user requests.

The reception unit can input information on a name, a dosage, a frequency of taking, a time of taking, and a side effect of the medicine. The reception unit provides, for example, a text field for inputting the name of the medicine. When inputting the name of the medicine, the user can use a function of auto-completing a candidate medicine name. In addition, the reception unit provides a numerical field for inputting the dosage. When inputting the dosage, the user can select a unit such as mg, ml, or the number of tablets. Furthermore, the reception unit provides a drop-down menu for inputting the frequency of taking or the time of taking. The user can select the frequency of taking from options such as three times a day or after every meal. In addition, the user can select the time of taking from options such as after breakfast or before bedtime. The reception unit provides a text area for inputting information on the side effect. The user can input a specific type or influence of the side effect. For example, information such as nausea, headache, or allergic reaction can be input. Accordingly, by inputting detailed information on the medicine, accuracy of analysis or adjustment is improved. Part or all of the above-described processing in the reception unit may be performed using, for example, AI, or may be performed without using AI. For example, the reception unit can input the medicine information input by the user to AI, and the AI can automatically complete the medicine name. Specifically, the present reception unit is composed of a user interface (UI) layer and a data processing layer, and executes validation and normalization processing of input data in real time. In the auto-complete function, a character prediction model using a recurrent neural network (RNN) or LSTM (Long Short-Term Memory) is incorporated, which receives a sequence of several characters input by the user as input, and outputs a probability distribution of a character string likely to come next, thereby dynamically generating a candidate list. In addition, in inputting the side effect information, a natural language processing model (for example, a BERT-based encoder) is adopted, which analyzes free description text input by the user, extracts specific symptom entities such as “nausea” and “headache” (Named Entity Recognition), and maps them to a code of a standardized medical term dictionary (MedDRA, etc.). At this time, input to the AI model is tokenized text data, and output is an extracted symptom label and a confidence score thereof. Furthermore, a function can be provided that acquires an image of a package or a tablet of the medicine with a camera using image recognition technology, and extracts an image feature amount using a convolutional neural network (CNN), thereby automatically identifying a type or a dose of the medicine. This provides a technical effect of preventing a mistake due to manual input, ensuring accuracy of data, and significantly reducing an input load on the user.

The analysis unit can be based on a case where a combination of specific medicines is dangerous or a case where a side effect is reduced by taking the medicine in a specific time zone. The analysis unit considers, for example, a case where a combination of specific medicines is dangerous. The analysis unit evaluates a risk of interaction based on component information of the medicine. For example, the analysis unit collates the component information of the medicine with a database and evaluates the risk of interaction. In addition, the analysis unit considers a case where a side effect is reduced by taking the medicine in a specific time zone. The analysis unit analyzes a timing of taking the medicine and proposes an optimal time of taking. For example, the analysis unit evaluates a case where a side effect is reduced by taking the medicine in a specific time zone based on the component information of the medicine. Accordingly, by considering the combination or timing of taking the medicine, safe and effective taking of the medicine becomes possible. Part or all of the above-described processing in the analysis unit may be performed using, for example, AI, or may be performed without using AI. For example, the analysis unit can input the component information of the medicine to AI, and the AI can automatically evaluate the risk of interaction. Specifically, the present analysis unit includes an inference engine cooperating with a large-scale drug interaction database, and executes an inference algorithm using a knowledge graph. The analysis unit constructs a graph structure having input plurality of medicine components as nodes and an interaction relationship therebetween as an edge, and detects a combination requiring contraindication or caution using a graph search algorithm. Furthermore, when an interaction prediction model using deep learning (for example, DeepDDI, etc.) is adopted as the AI model, a chemical structure descriptor (fingerprint) or target protein information of each medicine is input as a high-dimensional vector, and an interaction type (metabolic inhibition, competition, etc.) between medicine pairs and an occurrence probability thereof are output. In addition, in analysis of side effect reduction by time zone, simulation using gene expression data related to a biological clock (circadian rhythm) or a pharmacokinetic parameter (absorption rate constant, elimination half-life, etc.) is performed, and an optimal time window in which a blood concentration falls within a therapeutic range and falls below a side effect threshold is calculated. By this processing, an unknown interaction that cannot be determined by mere database collation or precise risk evaluation according to individual metabolic capacity becomes possible, contributing to prevention of medical accidents and optimization of drug therapy.

The adjustment unit can adjust the timing of taking the medicine based on a daily schedule of the user. The adjustment unit adjusts the timing of taking the medicine in consideration of, for example, the daily schedule of the user. When there is a medicine to be taken by the user after breakfast, the adjustment unit notifies the time of taking in consideration of a time of breakfast. In addition, the adjustment unit can also immediately readjust the schedule when the user changes a schedule, and notify the timing of taking the medicine based on a new schedule. For example, the adjustment unit cooperates with a calendar application of the user and reflects a change in schedule in real time. Accordingly, by adjusting the timing of taking the medicine according to the schedule of the user, convenience is improved. Part or all of the above-described processing in the adjustment unit may be performed using, for example, AI, or may be performed without using AI. For example, the adjustment unit can input schedule information of the user to AI, and the AI can automatically readjust the schedule. Specifically, the present adjustment unit is implemented as a mathematical optimization solver or a reinforcement learning agent, and solves a multi-objective optimization problem. As input data, a calendar event of the user (start time, end time, event type), a taking constraint of the medicine (within 30 minutes after meal, 8-hour interval, etc.), and a life pattern of the user (wake-up time, bedtime) are received. These data are modeled as slots on a time axis, and attributes such as “takeable”, “not takeable”, and “recommended” are given to each slot. The adjustment unit sets an objective function that maximizes convenience of the user (less interruption) or medication compliance (prevention of forgetting to take) while satisfying a constraint condition, and searches for an optimal schedule solution using a genetic algorithm or simulated annealing. When reinforcement learning is used, a state space is defined as a current schedule and a list of untaken medicines, an action space is defined as allocation of taking time of each medicine, and a reward is defined as a schedule compliance rate or feedback from the user (confirmation button press, etc.). This allows the system to immediately perform recalculation and present a new optimal solution even when a sudden schedule change occurs, thereby realizing dynamic and adaptive schedule management distinct from static alarm setting.

The notification unit can, when the user changes a schedule, immediately readjust the schedule and notify the timing of taking the medicine based on a new schedule. For example, when the user changes a schedule, the notification unit immediately readjusts the schedule and notifies the timing of taking the medicine based on a new schedule. The notification unit transmits a push notification to a smartphone of the user. In addition, the notification unit can also transmit a notification to a mail address of the user. Furthermore, the notification unit can also transmit a notification to a smart watch of the user. For example, when the user changes a schedule, the notification unit immediately readjusts the schedule and notifies the timing of taking the medicine based on a new schedule. Accordingly, by immediately responding to the schedule change and notifying the timing of taking the medicine, convenience of the user is improved. Part or all of the above-described processing in the notification unit may be performed using, for example, AI, or may be performed without using AI. For example, the notification unit can input schedule information of the user to AI, and the AI can automatically readjust the schedule and perform notification. Specifically, the present notification unit adopts an event-driven architecture, and operates using a schedule update event from the adjustment unit as a trigger. The notification unit collects a context of a user device (current location, movement state, screen on/off state, etc.) in real time, and inputs the information as an input vector to a context recognition model. This model determines an optimal timing and notification means (vibration pattern, notification sound, screen display) at which the user easily notices the notification and which does not disturb work, and outputs a control signal. For example, when it is determined that the user is in a meeting (based on calendar information or voice environment analysis), the notification unit selects only a weak vibration notification to the smart watch, and performs control to suppress a voice notification on the smartphone. In addition, natural language generation (NLG) technology is used for generation of a notification message, and a message suitable for a context such as “Since the meeting is over, let's take the medicine now” is dynamically generated, instead of mere notification of time. This provides a technical effect of reducing stress of the user due to mechanical notification and smoothly promoting transition to medication taking behavior.

The notification unit can notify the timing of taking based on side effect information about the medicine by reflecting the information in a daily activity. The notification unit notifies the timing of taking based on, for example, side effect information about the medicine by reflecting the information in a daily activity. When a specific medicine causes drowsiness, the notification unit notifies to take the medicine at night. In addition, when a side effect is reduced by taking a specific medicine together with a meal, the notification unit can also notify the medicine in accordance with a time of the meal. For example, the notification unit collates the side effect information of the medicine with a database and proposes an optimal timing of taking. Accordingly, by notifying the timing of taking in consideration of the side effect information, health management of the user is improved. Part or all of the above-described processing in the notification unit may be performed using, for example, AI, or may be performed without using AI. For example, the notification unit can input the side effect information of the medicine to AI, and the AI can automatically propose an optimal timing of taking. Specifically, the present notification unit cooperates with a side effect risk evaluation module, and manages a side effect profile (incidence, severity, time to onset) for each medicine as a multidimensional vector. The system categorizes a scheduled action of the user (driving, meeting, exercise, etc.) by a classification model, and calculates a degree of influence of the side effect on each action. For example, an inner product of a side effect vector of “drowsiness” and an action vector of “driving” is calculated, and when a risk score exceeds a threshold, the system proposes shifting the timing of taking to after the driving ends. At this time, input to the AI model is side effect metadata of the medicine and schedule data of the user, and output is a recommendation score and a risk warning label in each time slot. Furthermore, the system updates the model using past user feedback (record such as “I became sleepy after taking this medicine”) as learning data, and performs personalized notification control reflecting side effect sensitivity for each individual. This enables safety management suitable for a lifestyle of an individual, which cannot be covered only by general package insert information.

The notification unit can integrate information on medicines prescribed at a plurality of hospitals and support a patient having poor knowledge of medicines so that the patient can take the medicines securely. The notification unit integrates, for example, information on medicines prescribed at a plurality of hospitals and supports a patient having poor knowledge of medicines so that the patient can take the medicines securely. The notification unit centrally manages information on medicines prescribed at a plurality of hospitals using integration of electronic medical records or a sharing method of medicine information. For example, when the patient inputs information on medicines prescribed at a plurality of hospitals, the notification unit automatically detects a duplicate medicine or a risk of interaction. In addition, the notification unit provides medicine information so that the patient can easily understand even if the patient has poor knowledge of medicines. For example, the notification unit displays a concise description of an effect or a side effect of the medicine. Accordingly, by integrating information on medicines prescribed at a plurality of hospitals, the patient can take the medicine securely. Part or all of the above-described processing in the notification unit may be performed using, for example, AI, or may be performed without using AI. For example, the notification unit can input information on medicines prescribed at a plurality of hospitals to AI, and the AI can automatically integrate the information and evaluate a risk. Specifically, the present notification unit includes a data integration engine, parses data of heterogeneous formats (HL7, FHIR, proprietary format, etc.) provided from different medical institutions, and executes aggregation processing for converting the data into a unified internal data schema. At this time, using a natural language processing model, medicines of the same component registered with different names (generic drugs, etc.) are identified as the same entity, and duplicate prescription is detected. Input to the AI model is a plurality of prescription data sets, and output is an integrated medicine list and an alert list of detected duplication/interaction. Furthermore, a “Paraphrasing Model” that converts a technical medical term into a plain word is mounted, and for example, a description “induces somnolence by antihistamine action” is automatically converted into a user-friendly expression “may become sleepy” and displayed. In addition, it is also possible to adopt a configuration that ensures tamper resistance and traceability in data sharing between a plurality of medical institutions using blockchain technology or distributed ledger technology. This technically solves a problem of polypharmacy (multiple drug use) and produces an effect of complementing self-management ability of the patient.

The reception unit can estimate an emotion of the user and adjust an input method of medicine information based on the estimated emotion of the user. The reception unit estimates, for example, an emotion of the user and adjusts the input method of medicine information based on the estimated emotion of the user. When the user feels stress, the reception unit provides a simple interface and minimizes an input procedure. In addition, when the user is relaxed, the reception unit can also provide a detailed input option and propose a customizable input method. Furthermore, when the user is in a hurry, the reception unit prioritizes voice input so that the medicine information can be input quickly. For example, the reception unit captures an expression of the user with a camera and estimates an emotion using an emotion estimation algorithm. Estimation of the emotion is realized using an emotion estimation function using, for example, an emotion engine or generative AI. The generative AI is text generation AI (for example, LLM), multimodal generative AI, or the like, but is not limited to such an example. Accordingly, by adjusting the input method according to the emotion of the user, convenience of input is improved. Part or all of the above-described processing in the reception unit may be performed using, for example, AI, or may be performed without using AI. For example, the reception unit can input emotion data of the user to AI, and the AI can automatically adjust the input method. Specifically, the present reception unit is equipped with a multimodal emotion recognition model, and simultaneously receives a video frame from a camera (movement of facial muscles), a voice waveform from a microphone (tone, pitch, speech speed of voice), and an operation log of a touch panel (strength of tap, input speed, mistouch rate) as input. These input data are vectorized by feature extractors such as a convolutional neural network (CNN) and a recurrent neural network (RNN), respectively, combined in an integration layer, and then output as a probability distribution of emotion classes (“impatience”, “anger”, “calm”, “confusion”, etc.). The system transmits a control signal to a UI rendering engine based on an emotion class having the highest probability. For example, when “impatience” is detected, the system hides a decorative element on a screen and immediately switches to a “simple mode” in which only a mandatory input item is displayed largely. In addition, when “confusion” is detected, an interactive assistant bot is activated and voice guidance is started. In this way, by sensing an internal state of the user and dynamically optimizing an interface, reduction of input errors and improvement of user experience (UX) are realized.

The reception unit can analyze a past medicine taking history of the user and propose an optimal input method. The reception unit analyzes, for example, a past medicine taking history of the user and proposes an optimal input method. The reception unit automatically displays medicine information frequently input by the user in the past as a candidate. In addition, the reception unit can also preferentially propose an input method (voice, text, etc.) used by the user in the past. Furthermore, the reception unit can also predict and propose medicine information to be used in a specific time zone from the past medicine taking history of the user. For example, the reception unit collates the past medicine taking history of the user with a database and proposes an optimal input method. Accordingly, by analyzing the past medicine taking history, an optimal input method can be proposed. Part or all of the above-described processing in the reception unit may be performed using, for example, AI, or may be performed without using AI. For example, the reception unit can input the past medicine taking history of the user to AI, and the AI can automatically propose an optimal input method. Specifically, the present reception unit includes a time-series data analysis engine, and applies a prediction algorithm using collaborative filtering or a Markov chain model to a database accumulating an operation log and a taking history of the user. Input to the AI model is a user ID, a current time, location information, and a latest operation sequence, and output is a ranking list of medicine IDs likely to be selected next or a score of a recommended input modality (voice, camera, keyboard). For example, by learning a behavior pattern of the user using GRU (Gated Recurrent Unit) which is a type of recurrent neural network (RNN), a periodic pattern such as “inputting a specific antihypertensive agent on the morning of the first Monday of every month” is detected, and “zero-click input” is realized in which an input confirmation screen of the medicine is presented immediately after application activation at the corresponding date and time. In addition, a situation where the user frequently uses voice input in the past (for example, while moving) is learned as a context, and adaptive control such as automatically turning on a microphone is performed when an acceleration sensor indicates movement. This reduces the number of input operations to the limit and supports continuous use.

The reception unit can perform filtering based on a current health condition or lifestyle habit of the user when inputting the medicine information. The reception unit performs filtering based on, for example, a current health condition or lifestyle habit of the user when inputting the medicine information. The reception unit filters medicine information to be input based on information on a medicine currently taken by the user. In addition, the reception unit can also propose appropriate medicine information based on the lifestyle habit (meal, exercise, etc.) of the user. Furthermore, the reception unit can also filter medicine information to be input based on the health condition (allergy, past medical history, etc.) of the user. For example, the reception unit collates the health condition or lifestyle habit of the user with a database and proposes optimal medicine information. Accordingly, by performing filtering based on the health condition or lifestyle habit, appropriate medicine information can be input. Part or all of the above-described processing in the reception unit may be performed using, for example, AI, or may be performed without using AI. For example, the reception unit can input data of the health condition or lifestyle habit of the user to AI, and the AI can automatically perform filtering. Specifically, the present reception unit includes a filtering engine that maps a personal health record (PHR) of the user to a vector space, and performs similarity calculation or logical operation with a medicine vector serving as an input candidate. As input data, a past medical history, allergy information, current vital data (blood pressure, heart rate, etc.), and a life log (number of steps, sleep time) of the user are used, and a user context vector integrating these is generated. On the other hand, an attribute tag based on an indication or contraindication information is given to each medicine in the medicine database. The system determines an inappropriate medicine (contraindication, allergy correspondence, duplication, etc.) in the current user context using a classification algorithm such as a decision tree or a random forest, and excludes (filters) the medicine from an input candidate list or displays the medicine with a warning. For example, when the user has an attribute of “egg allergy”, at a time when a medicine containing an egg-derived component (lysozyme hydrochloride, etc.) is listed as an input candidate, the system immediately performs filtering and proposes an alternative medicine. Since this processing excludes an inappropriate option at an initial stage of input, the processing functions as a safety mechanism for preventing health damage due to erroneous input.

The reception unit can estimate an emotion of the user and determine a priority of medicine information to be input based on the estimated emotion of the user. The reception unit estimates, for example, an emotion of the user and determines a priority of medicine information to be input based on the estimated emotion of the user. When the user feels stress, the reception unit preferentially inputs important medicine information. In addition, when the user is relaxed, the reception unit can also input detailed medicine information. Furthermore, when the user is in a hurry, the reception unit can also preferentially input necessary minimum medicine information. For example, the reception unit captures an expression of the user with a camera and estimates an emotion using an emotion estimation algorithm. Estimation of the emotion is realized using an emotion estimation function using, for example, an emotion engine or generative AI. The generative AI is text generation AI (for example, LLM), multimodal generative AI, or the like, but is not limited to such an example. Accordingly, by determining the priority of medicine information to be input according to the emotion of the user, efficiency of input is improved. Part or all of the above-described processing in the reception unit may be performed using, for example, AI, or may be performed without using AI. For example, the reception unit can input emotion data of the user to AI, and the AI can automatically determine the priority of medicine information. Specifically, the present reception unit executes a weighting algorithm that multiplies an output score (arousal, valence) from an emotion recognition model by importance metadata (mandatory item, optional item, urgency) of medicine information. The system includes a layout engine that dynamically changes a display order or a display size for each field (medicine name, dose, remarks, etc.) of an input form. For example, when the emotion recognition model detects “high stress/high arousal (panic state)”, the system maximizes a priority of a highly urgent medicine category such as “heart medicine” or “anti-seizure”, displays the category largely at the top of the screen, and collapses and hides a detailed remarks column. Conversely, in a case of a “relaxed” state, a full list including input items such as supplements and preventive medicines is displayed to encourage recording of a health condition. This prioritization processing is performed by matching a state vector of the user and an item attribute vector, and realizes adaptive UI control for ensuring input of information most necessary at that moment while minimizing a cognitive load on the user.

The reception unit can preferentially input highly relevant medicine information based on geographical location information of the user when inputting the medicine information. The reception unit preferentially inputs, for example, highly relevant medicine information in consideration of geographical location information of the user when inputting the medicine information. When the user is in a specific area, the reception unit preferentially inputs information on a medicine generally prescribed in the area. In addition, when the user is traveling, the reception unit can also preferentially input information on a medicine prescribed at a medical institution of a travel destination. Furthermore, when the user is at home, the reception unit can also preferentially input information on a medicine prescribed at home in the past. For example, the reception unit collates the geographical location information of the user with a database and proposes highly relevant medicine information. Accordingly, by considering the geographical location information, highly relevant medicine information can be preferentially input. Part or all of the above-described processing in the reception unit may be performed using, for example, AI, or may be performed without using AI. For example, the reception unit can input the geographical location information of the user to AI, and the AI can automatically propose highly relevant medicine information. Specifically, the present reception unit includes a context inference engine that receives location information (latitude, longitude, altitude) acquired from GPS, Wi-Fi, Bluetooth beacon, or the like as input, and identifies a current location (home, hospital A, pharmacy B, travel destination C, etc.) of the user using geofencing technology or a point of interest (POI) database. The system holds a probabilistic model such as a Bayesian network that has learned a correlation between each place (location) and a medicine set input in the past. For example, when it is detected that the user is within a geofence of a “family pharmacy”, a recommendation score of a list of medicines having a history of being dispensed at the pharmacy in the past is raised and displayed at a higher rank of input candidates. In addition, when it is determined that the user is traveling abroad, a function is also provided that automatically switches to a database corresponding to a local language or medicine name and preferentially suggests information on over-the-counter medicines purchasable locally. This narrows down a search space utilizing context information of a place, and significantly streamlines an input operation of the user.

The reception unit can analyze a social media activity of the user and input related medicine information when inputting the medicine information. The reception unit analyzes, for example, a social media activity of the user and inputs related medicine information when inputting the medicine information. The reception unit inputs related medicine information based on health information shared by the user on social media. In addition, the reception unit can also input medicine information based on information of a medical professional followed by the user on social media. Furthermore, the reception unit can also input medicine information based on information of a health-related group in which the user participates on social media. For example, the reception unit collates the social media activity of the user with a database and proposes related medicine information. Accordingly, by analyzing the social media activity, related medicine information can be input. Part or all of the above-described processing in the reception unit may be performed using, for example, AI, or may be performed without using AI. For example, the reception unit can input the social media activity of the user to AI, and the AI can automatically propose related medicine information. Specifically, the present reception unit acquires unstructured data such as posted text, an image, a history of “likes”, and a follow list through an API of a social media platform based on permission of the user. The system analyzes these text data using a natural language processing (NLP) model, and extracts a keyword related to health (such as “severe headache” or “suffering from hay fever”) or an entity. Furthermore, sentiment analysis is performed to identify a current health trouble or concern of the user. For example, when the user repeats a post “I cannot sleep recently”, the system understands the context and recommends a sleep improvement medicine or a supplement as an input candidate. In addition, it is also possible to detect a medicine package shown in a photo posted by the user using image recognition technology, and automatically reflect the information in an input form. By this processing, input support of medicine information based on a potential need becomes possible even if the user does not search explicitly.

The analysis unit can estimate an emotion of the user and adjust a representation method of analysis based on the estimated emotion of the user. For example, the analysis unit estimates the emotion of the user and adjusts the representation method of analysis based on the estimated emotion of the user. When the user is nervous, the analysis unit provides a simple and highly visible analysis result. Also, when the user is relaxed, the analysis unit can provide a detailed analysis result. Furthermore, when the user is in a hurry, the analysis unit can provide an analysis result that captures the main points. For example, the analysis unit captures a facial expression of the user with a camera and estimates the emotion using an emotion estimation algorithm. The estimation of the emotion is realized using an emotion estimation function using, for example, an emotion engine or generative AI. The generative AI is a text generative AI (for example, LLM), a multimodal generative AI, or the like, but is not limited to such examples. Thereby, by adjusting the representation method of analysis according to the emotion of the user, understanding of the analysis result is improved. Part or all of the above-described processing in the analysis unit may be performed using, for example, AI, or may be performed without using AI. For example, the analysis unit can input emotion data of the user to the AI, and the AI can automatically adjust the representation method of analysis. Specifically, the present analysis unit includes a presentation generation module configured to select and generate a display template when presenting an analysis result (interaction risk, recommended schedule, etc.) to the user. This module receives an emotion vector (e.g., [nervous: 0.8, relaxed: 0.1, impatient: 0.1]) output from an emotion recognition model as an input, and determines display parameters such as granularity of information (level of detail), presence or absence of use of technical terms, and heavy use of graphs and icons, in accordance with a policy optimized by reinforcement learning. For example, when the user is in a high tension state, the system omits complex pharmacological explanations to reduce cognitive load, and generates a simple conclusion close to binary such as “dangerous” or “safe” and an intuitive UI using a large warning icon. On the other hand, if in a relaxed state, the system generates rich content including a blood concentration transition graph and a detailed text explanation regarding a mechanism of occurrence of a side effect. In this way, by dynamically changing not only the “content” of information but also “how to convey” it according to a psychological state of the user, acceptance and understanding of the information are maximized.

The analysis unit can adjust a level of detail of analysis based on an importance of the medicine during analysis. For example, the analysis unit adjusts the level of detail of analysis based on the importance of the medicine during analysis. The analysis unit provides a detailed analysis result for an important medicine. Also, the analysis unit can provide a concise analysis result for a general medicine. Furthermore, the analysis unit can provide an analysis result that calls for particular attention for a medicine with a strong side effect. For example, the analysis unit collates the importance of the medicine with a database and proposes an optimal level of detail of analysis. Thereby, by adjusting the level of detail of analysis based on the importance of the medicine, important information can be preferentially provided. Part or all of the above-described processing in the analysis unit may be performed using, for example, AI, or may be performed without using AI. For example, the analysis unit can input the importance of the medicine to the AI, and the AI can automatically adjust the level of detail of analysis. Specifically, the present analysis unit assigns indices such as “Life-Criticality Score” and “Therapeutic Index” to each medicine, and controls allocation of analysis resources and resolution of output information based on these. Input data is metadata of the medicine, and an output is a configuration tree of an analysis report. For example, for a high-risk medicine (a medicine with a high importance score) such as an anticancer drug or an immunosuppressant, the system performs a molecular-level interaction simulation or a detailed metabolic analysis considering genetic polymorphism, and generates a detailed report spanning several pages. On the other hand, for a low-risk medicine such as a vitamin supplement, the system performs only a basic combination check and displays only a summary of a few lines. This control logic is implemented by fuzzy logic or a rule-based system, and achieves both efficiency of calculation resources and modulation of alerting to the user.

The analysis unit can apply a different analysis algorithm according to a category of the medicine during analysis. For example, the analysis unit applies a different analysis algorithm according to the category of the medicine during analysis. For antibiotics, the analysis unit applies an algorithm that analyzes an effect on specific pathogenic bacteria. Also, for analgesics, the analysis unit can apply an algorithm that analyzes an effect according to a type of pain. Furthermore, for antidepressants, the analysis unit can apply an algorithm that analyzes an effect on a mental state. For example, the analysis unit collates the category of the medicine with a database and proposes an optimal analysis algorithm. Thereby, by applying the analysis algorithm according to the category of the medicine, accuracy of the analysis result is improved. Part or all of the above-described processing in the analysis unit may be performed using, for example, AI, or may be performed without using AI. For example, the analysis unit can input the category of the medicine to the AI, and the AI can automatically apply an optimal analysis algorithm. Specifically, the present analysis unit has an algorithm selection mechanism using a strategy pattern, and dynamically switches an analysis module to be executed using a drug efficacy classification code (ATC code, etc.) of the medicine as an input. For example, in the case of antibiotics, the system selects an algorithm that simulates a blood concentration maintenance time required for suppression of bacterial growth using a PK/PD (pharmacokinetics/pharmacodynamics) model. On the other hand, in the case of analgesics, the system selects a time-series matching algorithm for synchronizing a circadian rhythm of pain (pain is strong in the morning, strong at night, etc.) with a peak time of drug efficacy. Also, in the case of antidepressants or psychotropic drugs, the system applies a model that estimates a receptor occupancy rate of a neurotransmitter or an algorithm that evaluates a risk of withdrawal symptoms. In this way, by selectively using a dedicated mathematical model or AI model specialized for characteristics of the medicine, high-precision prediction and advice that cannot be obtained by general-purpose analysis become possible.

The analysis unit can estimate an emotion of the user and adjust a length of analysis based on the estimated emotion of the user. For example, the analysis unit estimates the emotion of the user and adjusts the length of analysis based on the estimated emotion of the user. When the user is in a hurry, the analysis unit provides a short analysis result that captures the main points. Also, when the user is relaxed, the analysis unit can provide a detailed analysis result. Furthermore, when the user is excited, the analysis unit can provide a visually stimulating analysis result. For example, the analysis unit captures a facial expression of the user with a camera and estimates the emotion using an emotion estimation algorithm. The estimation of the emotion is realized using an emotion estimation function using, for example, an emotion engine or generative AI. The generative AI is a text generative AI (for example, LLM), a multimodal generative AI, or the like, but is not limited to such examples. Thereby, by adjusting the length of analysis according to the emotion of the user, understanding of the analysis result is improved. Part or all of the above-described processing in the analysis unit may be performed using, for example, AI, or may be performed without using AI. For example, the analysis unit can input emotion data of the user to the AI, and the AI can automatically adjust the length of analysis. Specifically, the present analysis unit is equipped with an automatic summarization function using a Large Language Model (LLM), and dynamically controls the number of tokens (length) of text to be generated according to an emotion parameter of the user. As an input, raw data (text, numerical value) of the analysis result and an emotion label (e.g., “impatience”) from an emotion recognition module are received. The system uses prompt engineering technology to generate and execute an instruction to the LLM such as “Summarize only the conclusion within 30 characters for a user in an impatient state”. Conversely, when the user shows “interest” or “curiosity”, the instruction is switched to “Explain in about 500 characters including background knowledge and mechanism”. Also, it is possible to use an attention mechanism to identify a part having the highest importance for the user in the analysis result (for example, a part of side effect risk) and perform extractive summarization that preferentially leaves that part. Thereby, provision with an optimal amount of information matching a psychological acceptance capacity of the user is realized.

The analysis unit can determine a priority of analysis based on a timing of taking the medicine during analysis. For example, the analysis unit determines the priority of analysis based on the timing of taking the medicine during analysis. For a medicine whose timing of taking is near, the analysis unit preferentially provides an analysis result. Also, for a medicine whose timing of taking is far, the analysis unit can postpone providing an analysis result. Furthermore, for a medicine whose timing of taking is unknown, the analysis unit can ask the user for confirmation. For example, the analysis unit collates the timing of taking the medicine with a database and proposes an optimal priority of analysis. Thereby, by determining the priority of analysis based on the timing of taking the medicine, important information can be preferentially provided. Part or all of the above-described processing in the analysis unit may be performed using, for example, AI, or may be performed without using AI. For example, the analysis unit can input the timing of taking the medicine to the AI, and the AI can automatically determine the priority of analysis. Specifically, the present analysis unit incorporates a task scheduling algorithm, and manages an analysis request for each medicine in a priority queue. For calculation of the priority, a difference (remaining time) between a current time and a scheduled time of taking is used. The system assigns a high weight to an analysis task of a medicine with a short remaining time (for example, within one hour), and preferentially allocates calculation resources (CPU/GPU time). Thereby, it is guaranteed that an interaction check or display of precautions regarding a medicine immediately before taking is performed without delay. Also, a function can be provided in which an AI model is used to predict a “medicine highly likely to be taken next” from a past behavior pattern of the user, and analysis of the medicine is executed in advance in the background (prefetch processing). Thereby, an experience close to zero latency, such that the analysis result is displayed at the moment the user opens the application, is provided.

The analysis unit can adjust an order of analysis based on a relevance of the medicine during analysis. For example, the analysis unit adjusts the order of analysis based on the relevance of the medicine during analysis. The analysis unit preferentially provides an analysis result for a highly relevant medicine. In addition, the analysis unit can postpone providing an analysis result for a medicine with low relevance. Furthermore, the analysis unit can request the user for confirmation regarding a medicine whose relevance is unknown. For example, the analysis unit collates the relevance of the medicine with a database and proposes an optimal order of analysis. Thereby, by adjusting the order of analysis based on the relevance of the medicine, important information can be preferentially provided. Part or all of the above-described processing in the analysis unit may be performed using, for example, AI, or may be performed without using AI. For example, the analysis unit inputs the relevance of the medicine to the AI, and the AI can automatically adjust the order of analysis. Specifically, the analysis unit uses a Knowledge Graph in which dependency relationships and causal relationships between medicines are modeled. The system maps an input medicine list as nodes on a graph and determines an analysis order using a topological sort or a clustering algorithm. For example, in a case where there is a relationship in which an effect of a certain medicine A is enhanced by a medicine B, the system adjusts the order so as to analyze the medicine B first or analyze both simultaneously as a set. In addition, by identifying a medicine group (cluster) used for treating the same disease and analyzing them collectively, consistency as the entire treatment policy is evaluated. An input to an AI model is a set of medicine IDs, and an output is an optimized analysis execution sequence. This enables comprehensive analysis that captures individual medicines not as disjointed items but as a mutually related system.

The adjustment unit estimates an emotion of the user and can adjust a method of adjustment based on the estimated emotion of the user. For example, the adjustment unit estimates the emotion of the user and adjusts the method of adjustment based on the estimated emotion of the user. When the user feels stressed, the adjustment unit provides a simple adjustment method. In addition, when the user is relaxed, the adjustment unit can provide a detailed adjustment method. Furthermore, when the user is in a hurry, the adjustment unit can provide a quick adjustment method. For example, the adjustment unit captures a facial expression of the user with a camera and estimates the emotion using an emotion estimation algorithm. The estimation of the emotion is realized using an emotion estimation function using, for example, an emotion engine or a generative AI. The generative AI is a text generative AI (for example, LLM), a multimodal generative AI, or the like, but is not limited to such examples. Thereby, by adjusting the method of adjustment according to the emotion of the user, convenience of adjustment is improved. Part or all of the above-described processing in the adjustment unit may be performed using, for example, AI, or may be performed without using AI. For example, the adjustment unit inputs emotion data of the user to the AI, and the AI can automatically adjust the method of adjustment. Specifically, the adjustment unit has a policy network that controls behavior of a dialogue agent that proposes a schedule change. When an emotion recognition module detects “irritation” or “high load”, the adjustment unit shifts to a “one-tap adjustment” mode, eliminates complex options, presents only one best schedule plan recommended by the AI, and provides an interface that is completed only with YES/NO. On the other hand, if the user is in a state of “having room”, the adjustment unit presents a plurality of alternatives (Plan A, Plan B, Plan C) and provides an interactive Gantt chart UI that allows fine adjustment by drag and drop. This control is optimized by a reinforcement learning model that performs mapping between a user state space and a UI action space, and realizes schedule adjustment with a sense of satisfaction while minimizing a psychological burden on the user.

The adjustment unit can analyze a past schedule history of the user and select an optimal adjustment method during adjustment. For example, the adjustment unit analyzes the past schedule history of the user and selects the optimal adjustment method during adjustment. The adjustment unit proposes the optimal adjustment method based on a schedule used by the user in the past. In addition, the adjustment unit can propose an adjustment method suitable for a specific time zone from the past schedule history of the user. Furthermore, the adjustment unit can analyze the past schedule history of the user and propose the most efficient adjustment method. For example, the adjustment unit collates the past schedule history of the user with a database and proposes the optimal adjustment method. Thereby, by analyzing the past schedule history, the optimal adjustment method can be selected. Part or all of the above-described processing in the adjustment unit may be performed using, for example, AI, or may be performed without using AI. For example, the adjustment unit inputs the past schedule history of the user to the AI, and the AI can automatically select the optimal adjustment method. Specifically, the adjustment unit learns a life rhythm of the user and a change pattern of the schedule using time-series data mining technology. The system receives schedule data for the past several months (start/end times of events, change history, medicine taking record) as input, and extracts a “type (template)” of a schedule that is easy for the user to accept, using a sequence model such as LSTM or Transformer. For example, when the user has a pattern of “always taking a 30-minute relaxation time after dinner on weekdays”, the system sets a time of taking the medicine avoiding that time zone, or adjusts so as to prompt taking the medicine immediately before the relaxation time. In addition, the system also performs “negative feedback learning” in which an adjustment plan rejected by the user in the past (e.g., taking the medicine early in the morning) is learned so as not to make a similar proposal. This enables personalized adjustment that fits the user's preference as it is used more.

The adjustment unit can customize a means for adjustment based on a current living situation of the user during adjustment. For example, the adjustment unit customizes the means for adjustment based on the current living situation of the user during adjustment. When the user is busy, the adjustment unit provides a concise adjustment means. In addition, when the user is relaxed, the adjustment unit can provide a detailed adjustment means. Furthermore, when the user is participating in a specific event, the adjustment unit can provide an adjustment means tailored to the event. For example, the adjustment unit collates the current living situation of the user with a database and proposes an optimal adjustment means. Thereby, by customizing the means for adjustment based on the current living situation, accuracy of adjustment is improved. Part or all of the above-described processing in the adjustment unit may be performed using, for example, AI, or may be performed without using AI. For example, the adjustment unit inputs the current living situation of the user to the AI, and the AI can automatically propose the optimal adjustment means. Specifically, the adjustment unit includes an activity recognition model that integrates sensor data (acceleration, gyro, heart rate, environmental sound, calendar schedule) of a smartphone or a wearable device and estimates a current context of the user (such as “in a meeting”, “driving”, “sleeping”, “exercising”) in real time. Input data is multimodal time-series sensor data, and output is a probability distribution of activity classes. For example, when the system determines that the user is “driving”, the system invalidates an adjustment means involving a screen operation and switches to a mode that accepts only simple adjustment by a voice command. In addition, if the user is “in a meeting”, the system eliminates a notification sound and proposes fine adjustment of the schedule (e.g., delaying taking the medicine by 30 minutes) only by vibration notification to a smart watch. In this way, by providing an adjustment means physically and socially adapted to a situation in which the user is placed, a system operation considering safety and sociality is realized.

The adjustment unit estimates an emotion of the user and can determine a priority of adjustment based on the estimated emotion of the user. For example, the adjustment unit estimates the emotion of the user and determines the priority of adjustment based on the estimated emotion of the user. When the user feels stressed, the adjustment unit preferentially performs important adjustment. In addition, when the user is relaxed, the adjustment unit can perform detailed adjustment. Furthermore, when the user is in a hurry, the adjustment unit can preferentially perform necessary minimum adjustment. For example, the adjustment unit captures a facial expression of the user with a camera and estimates the emotion using an emotion estimation algorithm. The estimation of the emotion is realized using an emotion estimation function using, for example, an emotion engine or a generative AI. The generative AI is a text generative AI (for example, LLM), a multimodal generative AI, or the like, but is not limited to such examples. Thereby, by determining the priority of adjustment according to the emotion of the user, efficiency of adjustment is improved. Part or all of the above-described processing in the adjustment unit may be performed using, for example, AI, or may be performed without using AI. For example, the adjustment unit inputs emotion data of the user to the AI, and the AI can automatically determine the priority of adjustment. Specifically, the adjustment unit uses an emotion score as a weighting coefficient in a resolution logic when a plurality of schedule conflicts occur. The system holds a list of tasks requiring adjustment (taking medicine A, taking medicine B, implementation of schedule C), and “medical importance” and “psychological cost for user” are scored for each task. When the emotion recognition model indicates “high stress”, the system increases a weight of the “psychological cost” and preferentially generates an adjustment plan with less burden on the user (for example, reducing the number of times by combining times of taking the medicine). Conversely, if the user is in a “motivated” state, the system prioritizes strict time management (for example, strictly separating before and after meals) to maximize a medical effect. This optimization is mathematically executed by incorporating an emotion term into an evaluation function of linear programming or a genetic algorithm.

The adjustment unit can select an optimal adjustment method based on geographical location information of the user during adjustment. For example, the adjustment unit selects the optimal adjustment method based on the geographical location information of the user during adjustment. When the user is in a specific area, the adjustment unit selects an adjustment method based on information on medical institutions in the area. In addition, when the user is traveling, the adjustment unit can select an adjustment method based on information on medical institutions at a travel destination. Furthermore, when the user is at home, the adjustment unit can select an adjustment method used at home in the past. For example, the adjustment unit collates the geographical location information of the user with a database and proposes the optimal adjustment method. Thereby, by considering the geographical location information, the optimal adjustment method can be selected. Part or all of the above-described processing in the adjustment unit may be performed using, for example, AI, or may be performed without using AI. For example, the adjustment unit inputs the geographical location information of the user to the AI, and the AI can automatically propose the optimal adjustment method. Specifically, the adjustment unit integrates Location-Based Service (LBS) logic, predicts a “next action” from a current location, a destination, and a moving speed of the user, and reflects it in schedule adjustment. For example, when it is calculated from GPS data that the user is moving toward “home” and an estimated arrival time is 18:00, the system performs adjustment to automatically slide a time of taking the medicine after dinner to 18:30 or later. In addition, when movement to an area with a time difference is detected, the system applies an algorithm for gradual change (gradually adjusting to local time) of a schedule for taking the medicine in consideration of a deviation of a biological clock (jet lag), thereby preventing poor physical condition due to a sudden time change. For this processing, an arrival time prediction model by a Recurrent Neural Network (RNN) using movement trajectory data or the like is utilized.

The adjustment unit can analyze a social media activity of the user and propose a means for adjustment during adjustment. For example, the adjustment unit analyzes the social media activity of the user and proposes the means for adjustment during adjustment. The adjustment unit proposes a related adjustment means based on health information shared by the user on social media. In addition, the adjustment unit can propose an adjustment means based on information on a medical professional followed by the user on social media. Furthermore, the adjustment unit can propose an adjustment means based on information on a health-related group in which the user participates on social media. For example, the adjustment unit collates the social media activity of the user with a database and proposes the related adjustment means. Thereby, by analyzing the social media activity, the related adjustment means can be proposed. Part or all of the above-described processing in the adjustment unit may be performed using, for example, AI, or may be performed without using AI. For example, the adjustment unit inputs the social media activity of the user to the AI, and the AI can automatically propose the related adjustment means. Specifically, the adjustment unit performs social graph analysis and extracts a lifestyle (for example, “morning activity”, “fasting”, etc.) recommended by an influencer or a community trusted by the user. The system inputs such lifestyle information as a constraint condition to a schedule optimization engine. For example, when the user participates in a group recommending a “morning-oriented life”, the system makes a proposal to shift the schedule for taking the medicine to a morning type. In addition, when the user posts a schedule of a “drinking party” on an SNS, the system detects the event and automatically generates an adjustment plan to advance the time of taking the medicine or shift it to the next morning in order to avoid interaction with alcohol. At this time, a technology for extracting the date and time and nature (presence or absence of drinking, etc.) of the event from posted content using natural language processing is used.

The notification unit estimates an emotion of the user and can adjust a method of notification based on the estimated emotion of the user. For example, the notification unit estimates the emotion of the user and adjusts the method of notification based on the estimated emotion of the user. When the user is nervous, the notification unit performs notification in a calm tone. In addition, when the user is relaxed, the notification unit can perform notification in a bright tone. Furthermore, when the user is in a hurry, the notification unit can perform quick and concise notification. For example, the notification unit captures a facial expression of the user with a camera and estimates the emotion using an emotion estimation algorithm. The estimation of the emotion is realized using an emotion estimation function using, for example, an emotion engine or a generative AI. The generative AI is a text generative AI (for example, LLM), a multimodal generative AI, or the like, but is not limited to such examples. Thereby, by adjusting the method of notification according to the emotion of the user, convenience of notification is improved. Part or all of the above-described processing in the notification unit may be performed using, for example, AI, or may be performed without using AI. For example, the notification unit inputs emotion data of the user to the AI, and the AI can automatically adjust the method of notification. Specifically, the notification unit includes a style transfer model that dynamically generates and converts frequency characteristics and rhythm of a notification sound and a writing style of a notification message. When an emotion recognition module detects “tension” or “anxiety”, the system selects or generates a sound (ambient music or the like) with a low frequency and a relaxed rhythm that has an effect of lowering a heart rate as the notification sound. In addition, when a Text-to-Speech (TTS) engine is used, a voice quality is adjusted to a “calm low tone”. Conversely, if the user is in a state of “relaxation” or “boredom”, a slightly up-tempo and bright notification sound and a voice with intonation are selected to call attention. By this multimodal output control, intervention is performed such that the notification itself contributes to emotion regulation of the user.

The notification unit can refer to a past activity history of the user and select an optimal notification method during notification. For example, the notification unit refers to the past activity history of the user and selects the optimal notification method during notification. The notification unit proposes the optimal notification method based on a notification method favorably used by the user in the past. In addition, the notification unit can propose a notification method suitable for a specific time zone from the past activity history of the user. Furthermore, the notification unit can analyze the past activity history of the user and propose the most effective notification method. For example, the notification unit collates the past activity history of the user with a database and proposes the optimal notification method. Thereby, by referring to the past activity history, the optimal notification method can be selected. Part or all of the above-described processing in the notification unit may be performed using, for example, AI, or may be performed without using AI. For example, the notification unit inputs the past activity history of the user to the AI, and the AI can automatically propose the optimal notification method. Specifically, the notification unit learns a notification strategy that maximizes a reaction rate of the user to notification (open rate, time until confirmation of taking the medicine) using a reinforcement learning method such as a Multi-Armed Bandit algorithm. The system has a plurality of notification actions (arms) such as “sound only”, “vibration only”, “screen lighting only”, and “combination thereof”, and selects an action with the highest expected reward in a current situation based on past history data (pairs of context and reward). For example, if a tendency that “reaction to vibration notification is good in the morning on weekdays” is learned from past data, the system preferentially selects vibration notification in that time zone. In addition, for a notification method with a poor reaction, parameters are exploratorily changed to autonomously learn a better method.

The notification unit can customize a means for notification based on a current living situation of the user during notification. For example, the notification unit customizes the means for notification based on the current living situation of the user during notification. When the user is busy, the notification unit provides a concise notification means. In addition, when the user is relaxed, the notification unit can provide a detailed notification means. Furthermore, when the user is participating in a specific event, the notification unit can provide a notification means tailored to the event. For example, the notification unit collates the current living situation of the user with a database and proposes an optimal notification means. Thereby, by customizing the means for notification based on the current living situation, accuracy of notification is improved. Part or all of the above-described processing in the notification unit may be performed using, for example, AI, or may be performed without using AI. For example, the notification unit inputs the current living situation of the user to the AI, and the AI can automatically propose the optimal notification means. Specifically, the notification unit is equipped with a model for estimating “Interruptibility” of the user. This model receives an acceleration sensor of a smartphone, a microphone (noise level), a calendar, and an application usage status (whether playing a game, browsing, etc.) as input, and outputs a score from 0 to 1 indicating whether it is okay to send a notification to the user now, and if so, what degree of intensity is appropriate. For example, when it is determined that the user is concentrating on work (typing sound of PC is detected, smartphone is face down, etc.), the system suspends immediate voice notification and activates a “delayed notification” function that performs notification at a break in work (such as a moment when the smartphone is lifted). This realizes context-aware notification that reliably transmits information without hindering work efficiency of the user.

The notification unit estimates an emotion of the user and can determine a priority of notification based on the estimated emotion of the user. For example, the notification unit estimates the emotion of the user and determines the priority of notification based on the estimated emotion of the user. When the user feels stressed, the notification unit preferentially performs important notification. In addition, when the user is relaxed, the notification unit can perform detailed notification. Furthermore, when the user is in a hurry, the notification unit can preferentially perform necessary minimum notification. For example, the notification unit captures a facial expression of the user with a camera and estimates the emotion using an emotion estimation algorithm. The estimation of the emotion is realized using an emotion estimation function using, for example, an emotion engine or a generative AI. The generative AI is a text generative AI (for example, LLM), a multimodal generative AI, or the like, but is not limited to such examples. Thereby, by determining the priority of notification according to the emotion of the user, efficiency of notification is improved. Part or all of the above-described processing in the notification unit may be performed using, for example, AI, or may be performed without using AI. For example, the notification unit inputs emotion data of the user to the AI, and the AI can automatically determine the priority of notification. Specifically, the notification unit performs dynamic sorting based on an emotion score in a notification queue that manages a plurality of notification messages (medicine taking alert, health advice, battery warning, etc.). The system assigns attributes of “urgency” and “emotional impact” to each notification item. When the emotion recognition model detects “high stress”, the system executes a filtering logic that suppresses or postpones notification with high “emotional impact” (which may further irritate the user) and delivers only notification with extremely high “urgency” (life-threatening) with top priority. This prevents omission of transmission of critical information while protecting mental health of the user.

The notification unit can select an optimal notification method based on geographical location information of the user during notification. For example, the notification unit selects the optimal notification method based on the geographical location information of the user during notification. When the user is in a specific area, the notification unit selects a notification method based on information on medical institutions in the area. In addition, when the user is traveling, the notification unit can select a notification method based on information on medical institutions at a travel destination. Furthermore, when the user is at home, the notification unit can select a notification method used at home in the past. For example, the notification unit collates the geographical location information of the user with a database and proposes the optimal notification method. Thereby, by considering the geographical location information, the optimal notification method can be selected. Part or all of the above-described processing in the notification unit may be performed using, for example, AI, or may be performed without using AI. For example, the notification unit inputs the geographical location information of the user to the AI, and the AI can automatically propose the optimal notification method. Specifically, the notification unit has an environment recognition function linked with position information and automatically applies a silent mode (manner mode) according to a place. For example, when it is determined by matching between GPS information and a map database that the user is in an area where quietness is required such as a “movie theater” or a “library”, the system forcibly turns off voice notification and switches to only vibration notification to a smart watch. In addition, the system also has an event trigger function that fires a reminder notification asking “Did you forget to take your medicine?” at the moment when it is detected by a geofence that the user has returned to “home”. By this processing, notification can be performed by an optimal method that is easy for the user to notice and does not cause trouble to surroundings.

The notification unit can analyze a social media activity of the user and propose a means for notification during notification. For example, the notification unit analyzes the social media activity of the user and proposes the means for notification during notification. The notification unit proposes a related notification means based on health information shared by the user on social media. In addition, the notification unit can propose a notification means based on information on a medical professional followed by the user on social media. Furthermore, the notification unit can propose a notification means based on information on a health-related group in which the user participates on social media. For example, the notification unit collates the social media activity of the user with a database and proposes the related notification means. Thereby, by analyzing the social media activity, the related notification means can be proposed. Part or all of the above-described processing in the notification unit may be performed using, for example, AI, or may be performed without using AI. For example, the notification unit inputs the social media activity of the user to the AI, and the AI can automatically propose the related notification means. Specifically, the notification unit analyzes an SNS usage situation (active time zone, frequently used application) of the user and optimizes a notification channel. For example, when the user frequently uses a specific messaging application (LINE, Messenger, etc.), the system proposes sending notification via a bot through an API of the application. In addition, when the user makes a post seeking “encouragement” on an SNS, a phrase promoting motivation improvement such as “Let's do your best!” is added to a notification message. In this way, by performing notification in a form that blends into a digital environment with which the user comes into contact on a daily basis, an activation rate of the application is increased and compliance with taking the medicine is improved.

The system according to the embodiment is not limited to the above-described example, and various modifications are possible, for example, as follows. Specifically, the system adopts a module structure, and since each functional block (reception unit, analysis unit, adjustment unit, notification unit) is implemented as an independent microservice, it is possible to easily add, delete, or replace an individual function. In addition, the system can be deployed in any of cloud, edge, or hybrid environments, and a configuration can be flexibly changed according to a privacy requirement or a communication environment of the user. Furthermore, regarding an AI model, various implementation forms can be taken, such as an on-device AI method in which a learned model is downloaded from a server and inference is performed on a terminal side, and a method in which a model is updated while protecting privacy using Federated Learning.

The reception unit can be provided with a function of inputting a meal content of the user. For example, by inputting the meal content by the user, a timing of taking the medicine can be adjusted according to a meal. Furthermore, the reception unit can be provided with a function of inputting allergy information of the user. This enables selection of a medicine and adjustment of a timing of taking the medicine to avoid an allergic reaction. In addition, the reception unit can be provided with a function of inputting an exercise habit of the user. For example, by adjusting a timing of a medicine to be taken after exercise, an effect of the medicine can be maximized. Specifically, the reception unit is equipped with a meal image recognition AI, and automatically estimates calories, nutrients (carbohydrate, lipid, protein), and food ingredients from a photograph of a meal taken by the user. This estimation result (nutrient vector) is sent to the analysis unit and used for precise adjustment such as recommending taking the medicine after a meal with high lipid in order to increase an absorption rate of a fat-soluble medicine. In addition, regarding the exercise habit, the reception unit has a function of automatically importing activity amount data (number of steps, heart rate, calorie consumption) acquired from a wearable device and finely adjusting the timing of taking the medicine in consideration of a change in metabolism according to exercise intensity.

The analysis unit can use genetic information of the user for analysis. For example, based on the genetic information of the user, reactivity to a specific medicine can be evaluated, and an optimal medicine can be proposed. In addition, the analysis unit can also use lifestyle habit data of the user for analysis. For example, when there is a habit of smoking or drinking, selection of a medicine or adjustment of a timing of taking the medicine can be performed according to the habit. Furthermore, the analysis unit can also use a stress level of the user for analysis. For example, in a period of high stress, a medicine with few side effects can be preferentially proposed. Specifically, the analysis unit is provided with an analysis engine based on pharmacogenomics, and receives DNA sequence data (particularly genetic polymorphism related to drug-metabolizing enzyme CYP450) of the user as input. The system classifies the user into an “Ultra-rapid Metabolizer”, a “Poor Metabolizer”, or the like based on a genotype, and predicts a risk that an effect is insufficient or a side effect strongly appears with a standard dosage. In addition, regarding the lifestyle habit data, enzyme induction due to smoking or metabolic competition due to drinking is mathematically modeled, and a parameter of blood concentration simulation is corrected, thereby calculating an administration plan completely adapted to a constitution and a lifestyle habit of an individual.

The adjustment unit can adjust a timing of taking the medicine in consideration of a sleep pattern of the user. For example, when the user works a night shift, an effect of the medicine can be maximized by adjusting a timing of taking the medicine at night. In addition, the adjustment unit can also adjust the timing of taking the medicine in consideration of a travel schedule of the user. For example, when traveling to an area with a time difference, the timing of taking the medicine can be adjusted according to local time. Furthermore, the adjustment unit can also adjust the timing of taking the medicine in consideration of an exercise schedule of the user. For example, by adjusting a timing of taking the medicine before and after exercise, the effect of the medicine can be maximized. Specifically, the adjustment unit analyzes sleep stage data (REM sleep, non-REM sleep, awakening) acquired from a sleep meter or a smart watch, and estimates a phase of a circadian rhythm of the user. Based on knowledge of Chronopharmacology, the system sets the timing of taking the medicine according to a circadian time when sensitivity of a target organ is highest. For example, considering that cholesterol synthase is activated at night, a statin medicine is set before bedtime, but in the case of a night shift worker, automatic adjustment is performed so as to set it before sleep in the daytime. In addition, as a countermeasure against jet lag, an algorithm for generating a gradual shift plan of a schedule for taking the medicine in consideration of a secretion rhythm of melatonin is also installed.

The notification unit can notify the timing of taking the medicine in cooperation with a calendar application of the user. For example, notification of taking the medicine can be performed at an appropriate timing according to a schedule of the user. In addition, the notification unit can also notify the timing of taking the medicine in cooperation with a smart home device of the user. For example, notification can be performed by voice through a smart speaker. Furthermore, the notification unit can also notify taking the medicine at an appropriate timing after exercise in cooperation with a fitness tracker of the user. Specifically, the notification unit has a function as an IoT (Internet of Things) hub and cooperates with smart home appliances (lighting, speaker, television) in a home by a protocol such as MQTT. When it comes to a time of taking the medicine, the system performs environment control such as not only ringing a smartphone but also blinking lighting in a living room in a specific color (for example, a color of the medicine) or announcing “It is time for your medicine” from the smart speaker. In addition, in calendar cooperation, by performing notification aiming at “travel time” or “free time” of a schedule, a timing easy for the user to respond is pinpointed. This provides an ambient notification experience using the entire living space and prevents forgetting to take the medicine.

The notification unit estimates an emotion of the user and can adjust a content of notification based on the estimated emotion of the user. For example, when the user feels stressed, a notification content that allows relaxation is provided. In addition, when the user is relaxed, a detailed notification content can be provided. Furthermore, when the user is in a hurry, a concise notification content can be provided. Thereby, by adjusting the content of notification according to the emotion of the user, an effect of notification can be maximized. Specifically, the notification unit is provided with a message generation function using a Large Language Model (LLM), and rewrites a tone and manner of a notification text in real time based on input (emotion label, intensity) from an emotion recognition module. For example, for a user with high stress, instead of an imperative form “Please take your medicine”, a sympathetic and proposal-type message such as “Why don't you take a break and take your medicine? Deep breathing is also recommended” is generated. In addition, a mechanism is provided in which sentiment analysis is performed on the generated message, and delivery is performed after safety confirmation (guardrail) as to whether or not the current emotion of the user is rubbed the wrong way is performed.

The reception unit estimates an emotion of the user and can adjust an input method of medicine information based on the estimated emotion of the user. For example, when the user feels stressed, a simple interface is provided to minimize an input procedure. In addition, when the user is relaxed, detailed input options are provided, and a customizable input method can be proposed. Furthermore, when the user is in a hurry, voice input is prioritized so that the medicine information can be input quickly. Thereby, by adjusting the input method according to the emotion of the user, convenience of input is improved. Specifically, the reception unit uses a model for estimating “Cognitive Load” from a biological reaction (pupil diameter, skin conductance, etc.) or an operation behavior (tap pressure, scroll speed) of the user. When it is determined that the load is high, the system applies a “progressive disclosure” pattern that reduces an amount of information on a screen, and switches to a wizard-style UI that displays only one question at a time. Conversely, when the load is low, a dashboard-style UI with high listability is provided to support efficient input. This dynamic transformation of the UI is instantaneously executed in conjunction with component state management of a front-end framework (React, Vue, etc.).

The analysis unit can estimate an emotion of the user and adjust a representation method of analysis based on the estimated emotion of the user. For example, when the user is nervous, the analysis unit provides a simple and highly visible analysis result. Also, when the user is relaxed, the analysis unit can provide a detailed analysis result. Furthermore, when the user is in a hurry, the analysis unit can provide an analysis result focusing on main points. Thereby, by adjusting the representation method of analysis according to the emotion of the user, understanding of the analysis result is improved. Specifically, the present analysis unit controls a data visualization engine (Visualization Engine) and changes a type and a color scheme of a graph in accordance with the emotion. For example, for a user feeling “anxiety”, a color scheme based on a calming color such as blue or green is applied while avoiding a warning color such as red, and a simple bar graph or pictogram is displayed instead of a complex scatter plot. Also, a hierarchical structure of information is dynamically changed, and a layout is reconstructed so as to display a solution to an anxiety factor (such as “Please consult a doctor”) at the top.

The adjustment unit can estimate an emotion of the user and adjust a method of adjustment based on the estimated emotion of the user. For example, when the user feels stress, the adjustment unit provides a simple adjustment method. Also, when the user is relaxed, the adjustment unit can provide a detailed adjustment method. Furthermore, when the user is in a hurry, the adjustment unit can provide a quick adjustment method. Thereby, by adjusting the method of adjustment according to the emotion of the user, convenience of adjustment is improved. Specifically, the present adjustment unit switches an interaction model with the user between a “leading type” and a “passive type”. When the user is under stress, the system becomes the “leading type”, presents an optimal solution determined by AI, and asks only for approval. On the other hand, in a relaxed state, the system becomes the “passive type” and provides a sandbox environment where the user can freely manipulate parameters (time, order). This switching is managed by a state machine (State Machine), and mode transition is performed seamlessly according to a change in an emotion score.

The notification unit can estimate an emotion of the user and adjust a method of notification based on the estimated emotion of the user. For example, when the user is nervous, the notification unit performs notification in a calm tone. Also, when the user is relaxed, the notification unit can perform notification in a bright tone. Furthermore, when the user is in a hurry, the notification unit can perform quick and concise notification. Thereby, by adjusting the method of notification according to the emotion of the user, convenience of notification is improved. Specifically, the present notification unit includes a driver generating a waveform pattern of haptic feedback (tactile notification) and changes a texture of vibration in accordance with the emotion. At the time of nervousness, the notification unit generates vibration of a slow beat pattern like a heartbeat and aims for an effect of calming the user. When the user is in a hurry, the notification unit attracts attention immediately with vibration of a short and sharp staccato pattern. These patterns are selected from a library designed based on psychophysical experimental data.

The reception unit can analyze a past medicine taking history of the user and propose an optimal input method. For example, the reception unit automatically displays medicine information frequently input by the user in the past as a candidate. Also, the reception unit can preferentially propose an input method (voice, text, etc.) used by the user in the past. Furthermore, the reception unit can predict and propose medicine information to be used in a specific time zone from the past medicine taking history of the user. Thereby, by analyzing the past medicine taking history, the optimal input method can be proposed. Specifically, the present reception unit constructs an input behavior model for each user and is equipped with a predictive input engine (Predictive Input Engine) that predicts a word or a numerical value having a high probability of being input next. This engine performs context-dependent prediction from input history data using an N-gram model or a neural language model. For example, the engine learns that there is a high probability that a dosage of “1 tablet” follows after inputting “Loxonin” from the past history, and presets “1” in a numerical field. Also, the engine learns a selection probability of an input device and performs UI optimization such as displaying a voice input button largely in a specific time zone (e.g., commuting time).

A flow of processing of Example of the Embodiment will be briefly described below. Specifically, a data processing pipeline in the present system is composed of a series of phases including data input, preprocessing, analysis/inference, optimization, and output/notification, and each phase is executed asynchronously and in parallel. Each step shown below is managed by a state machine inside the system and is implemented as strict transaction processing in which a data consistency check and log recording are performed in each state transition.

Step 1: The reception unit inputs information on a medicine to be taken by the user. The medicine to be taken by the user includes a medicine name, an ingredient, a dosage, a method of taking, a side effect, and the like. The reception unit provides a text field for inputting the medicine name, a numerical field for inputting the dosage, and a drop-down menu for inputting a frequency of taking and a time of taking. Also, the reception unit has a function of automatically completing a candidate medicine name when the user inputs the name of the medicine. Step 2: The analysis unit analyzes a combination or timing of the medicine based on the information input by the reception unit. The analysis unit considers a case where a combination of specific medicines is dangerous or a case where a side effect is reduced by taking the medicine in a specific time zone. The analysis unit evaluates a risk of interaction based on ingredient information of the medicine and proposes an optimal time of taking. Step 3: The adjustment unit adjusts a timing of taking the medicine based on information analyzed by the analysis unit. The adjustment unit adjusts the timing of taking the medicine in consideration of a daily schedule of the user, and immediately readjusts the schedule when the user changes a plan. The adjustment unit cooperates with a calendar app of the user and reflects a change in the plan in real time. Step 4: The notification unit performs notification of the timing of taking the medicine based on the timing adjusted by the adjustment unit. The notification unit transmits a push notification to a smartphone of the user and also transmits a notification to an email address or a smartwatch of the user. When the user changes a plan, the notification unit immediately readjusts the schedule and notifies the timing of taking the medicine based on a new schedule. Specifically, in Step 1, the reception unit converts input unstructured data (text, voice, image) into structured data (JSON, XML, etc.) and persists the data to a database. At this time, validation logic for detecting data loss or an abnormal value runs. In Step 2, the analysis unit fetches the latest medicine data from the database and inputs the data to an inference engine to calculate a risk score and a recommended time frame. This calculation is performed at high speed by parallel arithmetic using a GPU. In Step 3, the adjustment unit merges an analysis result and the latest schedule of the user (acquired from Google Calendar API, etc.) and generates a final schedule object by solving a constraint satisfaction problem. In Step 4, the notification unit transmits a push request to a notification service (APNs, FCM, etc.) of each device based on the generated schedule. This series of flows is orchestrated by messaging between microservices to ensure real-time performance.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of a plurality of elements including the above-described reception unit, analysis unit, adjustment unit, and notification unit is implemented by, for example, at least one of a smart device 14 and a data processing device 12. For example, the reception unit is implemented by a control unit 46A of the smart device 14 and inputs information on a medicine to be taken by a user. The analysis unit is implemented by, for example, a specific processing unit 290 of the data processing device 12 and analyzes a combination or timing of the medicine based on the input information. The adjustment unit is implemented by, for example, the specific processing unit 290 of the data processing device 12 and adjusts a timing of taking the medicine based on the analyzed information. The notification unit is implemented by, for example, the control unit 46A of the smart device 14 and performs notification of the timing of taking the medicine based on the adjusted timing. The correspondence relationship between each unit and the device or the control unit is not limited to the above-described example, and various modifications are possible.

### Second Embodiment

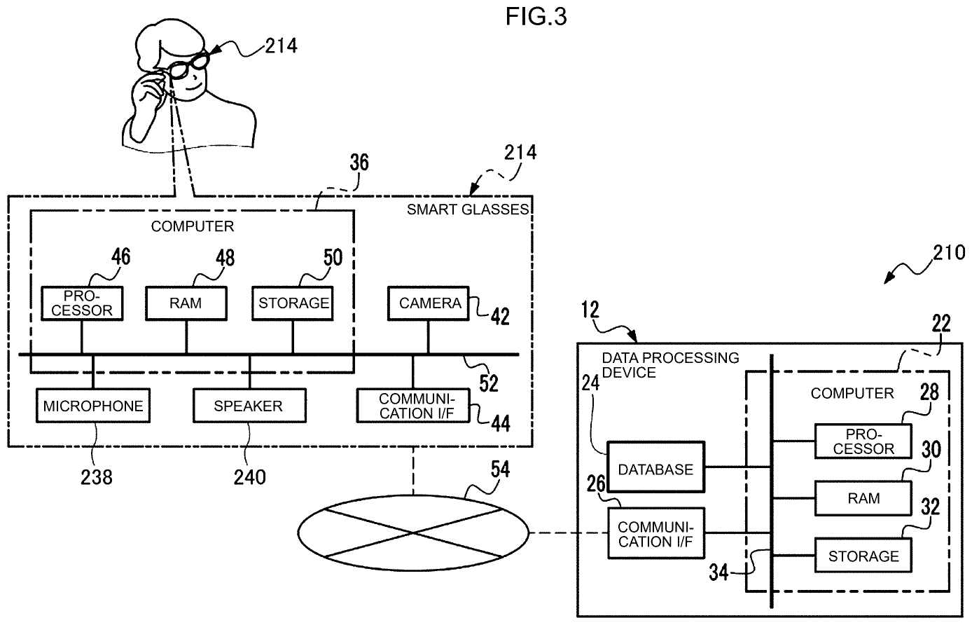

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

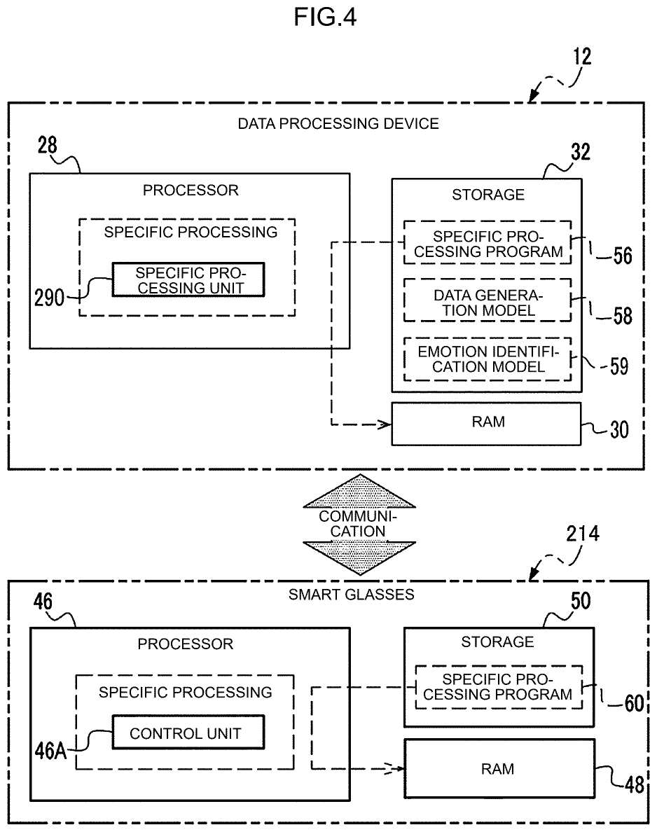

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of a plurality of elements including the above-described reception unit, analysis unit, adjustment unit, and notification unit is implemented by, for example, at least one of smart glasses 214 and the data processing device 12. For example, the reception unit is implemented by a control unit 46A of the smart glasses 214 and inputs information on a medicine to be taken by a user. The analysis unit is implemented by, for example, the specific processing unit 290 of the data processing device 12 and analyzes a combination or timing of the medicine based on the input information. The adjustment unit is implemented by, for example, the specific processing unit 290 of the data processing device 12 and adjusts a timing of taking the medicine based on the analyzed information. The notification unit is implemented by, for example, the control unit 46A of the smart glasses 214 and performs notification of the timing of taking the medicine based on the adjusted timing. The correspondence relationship between each unit and the device or the control unit is not limited to the above-described example, and various modifications are possible.

### Third Embodiment

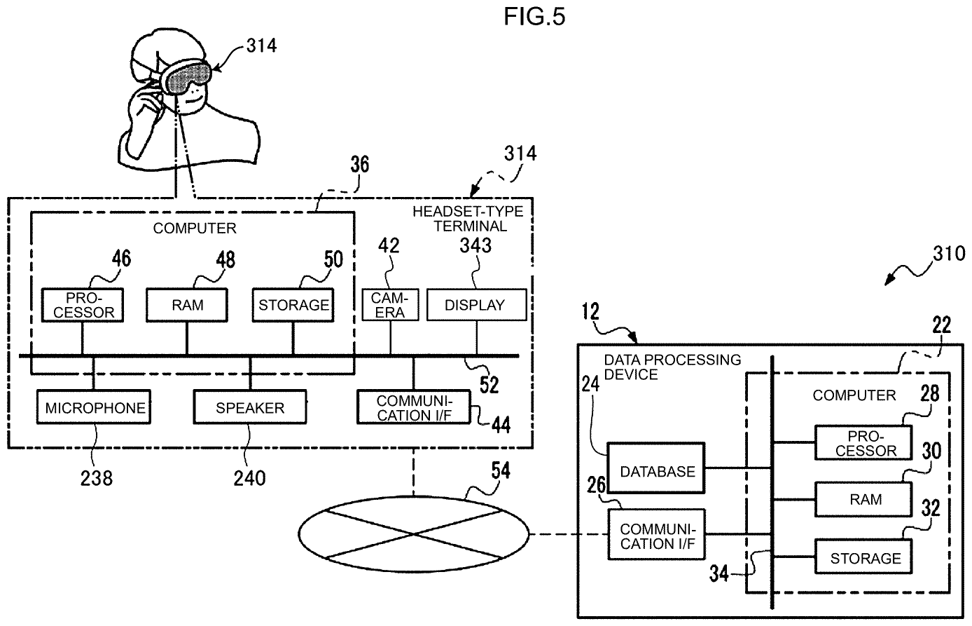

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

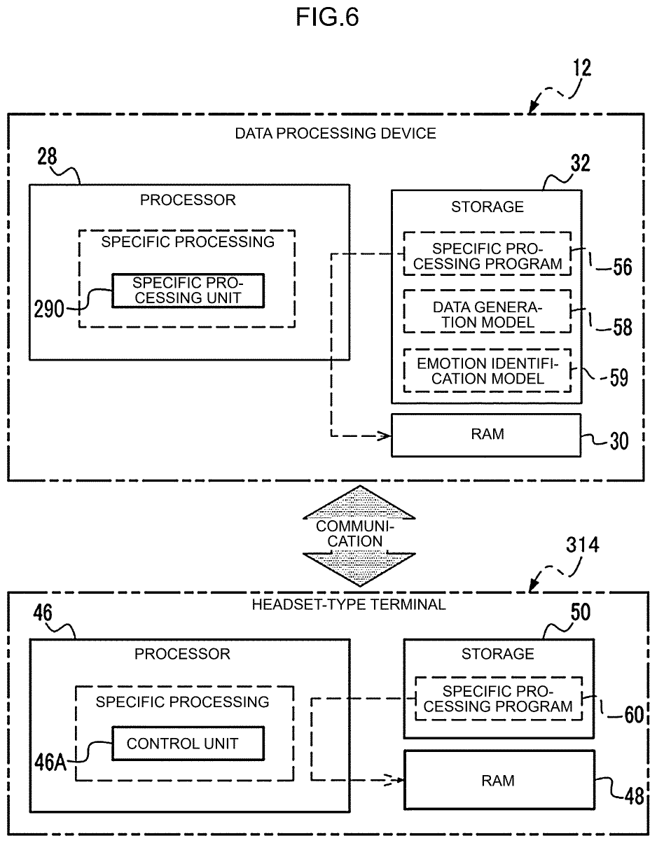

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of a plurality of elements including the above-described reception unit, analysis unit, adjustment unit, and notification unit is implemented by, for example, at least one of a headset-type terminal 314 and the data processing device 12. For example, the reception unit is implemented by a control unit 46A of the headset-type terminal 314 and inputs information on a medicine to be taken by a user. The analysis unit is implemented by, for example, the specific processing unit 290 of the data processing device 12 and analyzes a combination or timing of the medicine based on the input information. The adjustment unit is implemented by, for example, the specific processing unit 290 of the data processing device 12 and adjusts a timing of taking the medicine based on the analyzed information. The notification unit is implemented by, for example, the control unit 46A of the headset-type terminal 314 and performs notification of the timing of taking the medicine based on the adjusted timing. The correspondence relationship between each unit and the device or the control unit is not limited to the above-described example, and various modifications are possible.

### Fourth Embodiment

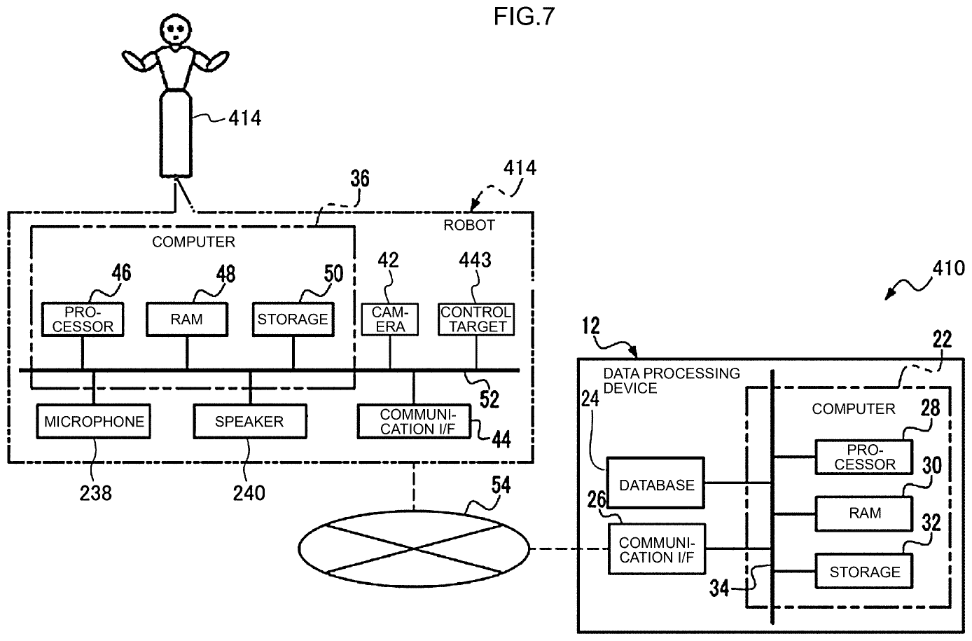

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

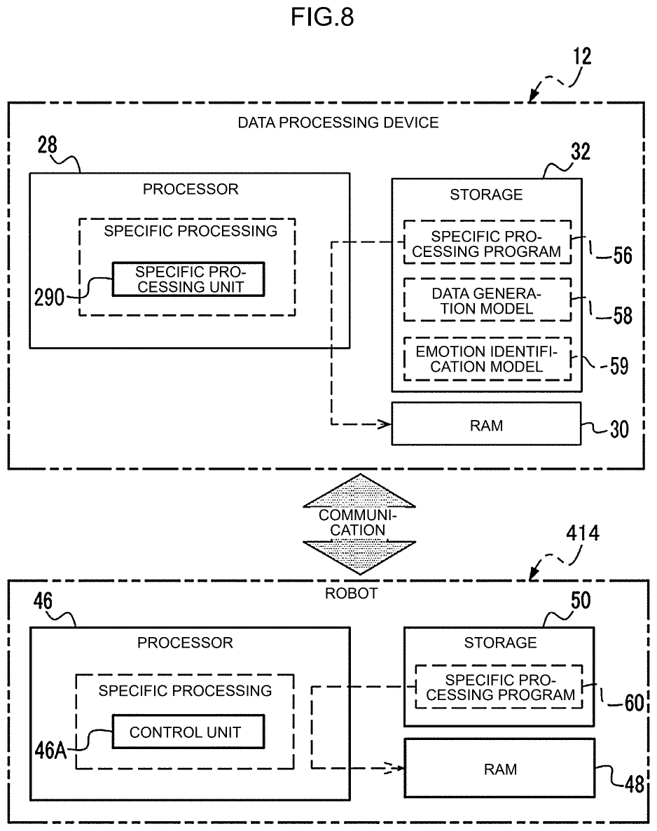

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of a plurality of elements including the above-described reception unit, analysis unit, adjustment unit, and notification unit is implemented by, for example, at least one of a robot 414 and the data processing device 12. For example, the reception unit is implemented by a control unit 46A of the robot 414 and inputs information on a medicine to be taken by a user. The analysis unit is implemented by, for example, the specific processing unit 290 of the data processing device 12 and analyzes a combination or timing of the medicine based on the input information. The adjustment unit is implemented by, for example, the specific processing unit 290 of the data processing device 12 and adjusts a timing of taking the medicine based on the analyzed information. The notification unit is implemented by, for example, the control unit 46A of the robot 414 and performs notification of the timing of taking the medicine based on the adjusted timing. The correspondence relationship between each unit and the device or the control unit is not limited to the above-described example, and various modifications are possible.

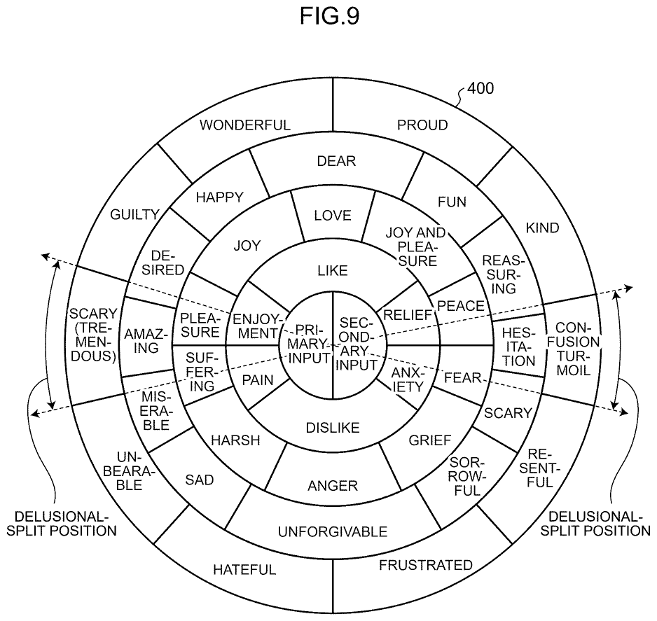

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

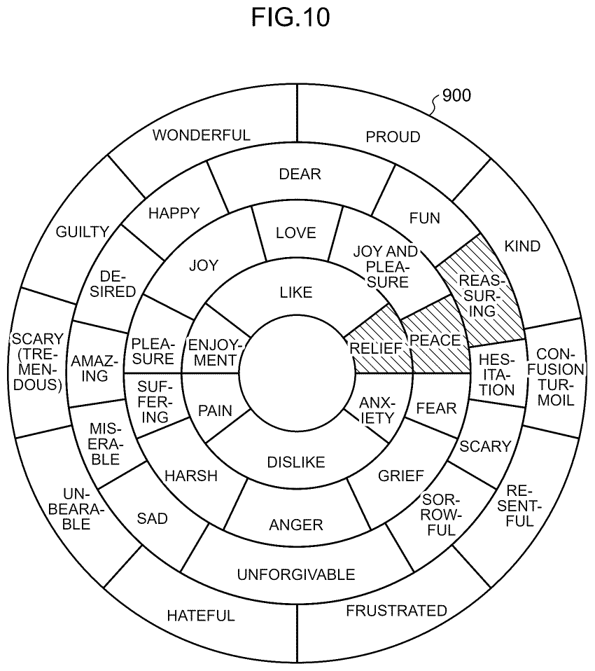

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: a reception unit configured to input information on a medicine to be taken by a user; an analysis unit configured to analyze a combination or timing of the medicine based on the information input by the reception unit; an adjustment unit configured to adjust a timing of taking the medicine based on information analyzed by the analysis unit; and a notification unit configured to perform notification of the timing of taking the medicine based on the timing adjusted by the adjustment unit.
(Supplementary Note 2) The system according to Supplementary Note 1, wherein the reception unit is configured to input information on a name, a dosage, a frequency of taking, a time of taking, and a side effect of the medicine.
(Supplementary Note 3) The system according to Supplementary Note 1, wherein the analysis unit is based on a case where a combination of specific medicines is dangerous or a case where a side effect is reduced by taking the medicine in a specific time zone.
(Supplementary Note 4) The system according to Supplementary Note 1, wherein the adjustment unit is configured to adjust the timing of taking the medicine based on a daily schedule of the user.
(Supplementary Note 5) The system according to Supplementary Note 1, wherein the notification unit is configured to, when the user changes a schedule, immediately readjust the schedule and notify the timing of taking the medicine based on a new schedule.
(Supplementary Note 6) The system according to Supplementary Note 1, wherein the notification unit is configured to notify the timing of taking based on side effect information about the medicine by reflecting the information in a daily activity.
(Supplementary Note 7)The system according to Supplementary Note 1, wherein the notification unit is configured to integrate information on medicines prescribed at a plurality of hospitals and support a patient having poor knowledge of medicines so that the patient can take the medicines securely.
(Supplementary Note 8) The system according to Supplementary Note 1, wherein the reception unit is configured to estimate an emotion of the user and adjust an input method of medicine information based on the estimated emotion of the user.
(Supplementary Note 9) The system according to Supplementary Note 1, wherein the reception unit is configured to analyze a past medicine taking history of the user and propose an optimal input method.
(Supplementary Note 10) The system according to Supplementary Note 1, wherein the reception unit is configured to perform filtering based on a current health condition or lifestyle habit of the user when inputting the medicine information.
(Supplementary Note 11) The system according to Supplementary Note 1, wherein the reception unit is configured to estimate an emotion of the user and determine a priority of medicine information to be input based on the estimated emotion of the user.
(Supplementary Note 12) The system according to Supplementary Note 1, wherein the reception unit is configured to preferentially input highly relevant medicine information based on geographical location information of the user when inputting the medicine information.
(Supplementary Note 13) The system according to Supplementary Note 1, wherein the reception unit is configured to analyze a social media activity of the user and input related medicine information when inputting the medicine information.
   (Supplementary Note 14) The system according to Supplementary Note 1, wherein the analysis unit is configured to estimate an emotion of the user and adjust a representation method of analysis based on the estimated emotion of the user.
(Supplementary Note 15) The system according to Supplementary Note 1, wherein the analysis unit is configured to adjust a level of detail of analysis based on an importance of the medicine during analysis.
(Supplementary Note 16) The system according to Supplementary Note 1, wherein the analysis unit is configured to apply a different analysis algorithm according to a category of the medicine during analysis.
(Supplementary Note 17) The system according to Supplementary Note 1, wherein the analysis unit is configured to estimate an emotion of the user and adjust a length of analysis based on the estimated emotion of the user.
(Supplementary Note 18) The system according to Supplementary Note 1, wherein the analysis unit is configured to determine a priority of analysis based on a timing of taking the medicine during analysis.
(Supplementary Note 19) The system according to Supplementary Note 1, wherein the analysis unit is configured to adjust an order of analysis based on a relevance of the medicine during analysis.
(Supplementary Note 20) The system according to Supplementary Note 1, wherein the adjustment unit is configured to estimate an emotion of the user and adjust a method of adjustment based on the estimated emotion of the user.
(Supplementary Note 21) The system according to Supplementary Note 1, wherein the adjustment unit is configured to analyze a past schedule history of the user and select an optimal adjustment method during adjustment.
(Supplementary Note 22) The system according to Supplementary Note 1, wherein the adjustment unit is configured to customize a means for adjustment based on a current living situation of the user during adjustment.
(Supplementary Note 23) The system according to Supplementary Note 1, wherein the adjustment unit is configured to estimate an emotion of the user and determine a priority of adjustment based on the estimated emotion of the user.
(Supplementary Note 24) The system according to Supplementary Note 1, wherein the adjustment unit is configured to select an optimal adjustment method based on geographical location information of the user during adjustment.
(Supplementary Note 25) The system according to Supplementary Note 1, wherein the adjustment unit is configured to analyze a social media activity of the user and propose a means for adjustment during adjustment.
(Supplementary Note 26) The system according to Supplementary Note 1, wherein the notification unit is configured to estimate an emotion of the user and adjust a method of notification based on the estimated emotion of the user.
(Supplementary Note 27) The system according to Supplementary Note 1, wherein the notification unit is configured to refer to a past activity history of the user and select an optimal notification method during notification.
(Supplementary Note 28) The system according to Supplementary Note 1, wherein the notification unit is configured to customize a means for notification based on a current living situation of the user during notification.
(Supplementary Note 29) The system according to Supplementary Note 1, wherein the notification unit is configured to estimate an emotion of the user and determine a priority of notification based on the estimated emotion of the user.
(Supplementary Note 30) The system according to Supplementary Note 1, wherein the notification unit is configured to select an optimal notification method based on geographical location information of the user during notification.
(Supplementary Note 31) The system according to Supplementary Note 1, wherein the notification unit is configured to analyze a social media activity of the user and propose a means for notification during notification.

What is claimed is:

1. A system comprising:
circuitry configured to:
receive, via a packet-switched network from a client terminal, a first data packet comprising a plurality of attribute vectors, each attribute vector corresponding to a respective item identifier;
extract, from the plurality of attribute vectors, a set of pairwise feature combinations and input the set of pairwise feature combinations into a graph neural network to generate an interaction risk vector indicating a degree of incompatibility for each pairwise feature combination;
generate, by inputting the interaction risk vector and a time-series constraint vector received from the client terminal into a reinforcement learning model, an output vector representing an optimized allocation of time slots that minimizes a cumulative risk value derived from the interaction risk vector; and
transmit, via the packet-switched network to the client terminal, a response data packet comprising the output vector.

2. The system according to claim 1, wherein the plurality of attribute vectors comprise attribute data indicating a name, a dosage, a frequency of consumption, a designated time of consumption, and a side effect profile for each of a plurality of medicines to be taken by the user.

3. The system according to claim 1, wherein the circuitry is further configured to determine, based on the interaction risk vector, whether a combination of items corresponding to two or more of the attribute vectors exceeds a danger threshold, and to identify a time zone in which a side effect associated with a particular item is reduced.

4. The system according to claim 1, wherein the time-series constraint vector comprises daily schedule data of the user, and the circuitry is further configured to compute the optimized allocation of time slots by correlating each time slot with an event in the daily schedule data.

5. The system according to claim 1, wherein the circuitry is further configured to, upon receiving an updated time-series constraint vector indicating a schedule change of the user, recompute the output vector and transmit an updated response data packet to the client terminal.

6. The system according to claim 1, wherein the circuitry is further configured to retrieve, from a database, side effect metadata associated with an item corresponding to one of the attribute vectors, classify a scheduled activity of the user using a classification model, and adjust the output vector such that a time slot for the item avoids a time period in which the side effect impairs the scheduled activity.

7. The system according to claim 1, wherein the circuitry is further configured to receive, from the client terminal, a plurality of data sets originating from different prescribing institutions, identify duplicate items across the plurality of data sets by applying a natural language processing model to normalize item names registered with different designations, and generate a unified item list with a duplication alert.

8. The system according to claim 1, wherein the circuitry is further configured to estimate an emotion of the user by applying an emotion identification model to sensor data received from the client terminal, and to adjust a configuration of an input interface rendered on the client terminal based on the estimated emotion.

9. The system according to claim 1, wherein the circuitry is further configured to analyze a past input history of the user stored in a database and generate a predicted input candidate list by applying a recurrent neural network to the past input history, and to transmit the predicted input candidate list to the client terminal.

10. The system according to claim 1, wherein the circuitry is further configured to receive, from the client terminal, user profile data indicating a current health condition or lifestyle habit of the user, and to filter the plurality of attribute vectors by excluding attribute vectors corresponding to items contraindicated for the current health condition using a classification algorithm.

11. The system according to claim 1, wherein the circuitry is further configured to estimate an emotion of the user by applying an emotion identification model to sensor data received from the client terminal, and to adjust a granularity of data included in the response data packet based on the estimated emotion such that a simplified output is generated when a stress level exceeds a threshold.

12. The system according to claim 1, wherein the circuitry is further configured to assign an importance score to each of the plurality of attribute vectors based on metadata stored in a database, and to allocate a higher computational resource to generating the interaction risk vector for attribute vectors having a higher importance score.

13. The system according to claim 1, wherein the circuitry is further configured to classify each item identifier into a category and to select, from a plurality of analysis algorithms, a category-specific analysis algorithm for generating the interaction risk vector corresponding to the classified category.

14. The system according to claim 1, wherein the circuitry is further configured to estimate an emotion of the user by applying an emotion identification model to sensor data received from the client terminal, and to determine a priority of recomputing the output vector based on the estimated emotion such that a simplified recomputation is performed when the estimated emotion indicates a high stress state.

15. The system according to claim 1, wherein the circuitry is further configured to receive, from the client terminal, geographical location information of the user, and to adjust the output vector based on a time zone difference associated with the geographical location information by applying a gradual shift algorithm to the optimized allocation of time slots.

16. The system according to claim 1, wherein the circuitry is further configured to retrieve, from a database, past schedule data of the user and apply a sequence model to the past schedule data to extract a schedule pattern of the user, and to constrain the reinforcement learning model using the extracted schedule pattern such that the output vector conforms to a learned preference of the user.

17. The system according to claim 1, wherein the circuitry is further configured to transmit the response data packet to a plurality of devices associated with the user via an Internet of Things protocol, and to select, for each of the plurality of devices, a notification modality based on a device type and a current context of the user determined from sensor data.

18. A system comprising:
a communication interface coupled to a packet-switched network;
a processor;
a random-access memory; and
a memory storing a graph neural network, a reinforcement learning model, and an emotion identification model, wherein the processor is configured to read and execute instructions from the memory on the random-access memory to function as circuitry configured to:
receive, via the communication interface from a client terminal, a first data packet comprising a plurality of attribute vectors, each attribute vector corresponding to a respective item identifier and indicating attribute information associated with a consumable item designated by a user;
extract, from the plurality of attribute vectors, a set of pairwise feature combinations and input the set of pairwise feature combinations into the graph neural network to generate an interaction risk vector indicating a degree of incompatibility for each pairwise feature combination;
generate, by inputting the interaction risk vector and a time-series constraint vector received via the communication interface from the client terminal into the reinforcement learning model, an output vector representing an optimized allocation of time slots that minimizes a cumulative risk value derived from the interaction risk vector;
estimate an emotion of the user by applying the emotion identification model to sensor data received via the communication interface from the client terminal;
adjust a notification parameter included in a response data packet based on the estimated emotion; and
transmit, via the communication interface to the client terminal, the response data packet comprising the output vector and the adjusted notification parameter.

19. The system according to claim 18, wherein the memory further stores a data generation model obtained by performing deep learning on a neural network, and the circuitry is further configured to generate, by inputting the interaction risk vector and the estimated emotion into the data generation model, a natural language notification message having a tone adjusted based on the estimated emotion, and to include the natural language notification message in the response data packet.

20. A method performed by circuitry of a system, the method comprising:
receiving, via a packet-switched network from a client terminal, a first data packet comprising a plurality of attribute vectors, each attribute vector corresponding to a respective item identifier;
extracting, from the plurality of attribute vectors, a set of pairwise feature combinations and inputting the set of pairwise feature combinations into a graph neural network to generate an interaction risk vector indicating a degree of incompatibility for each pairwise feature combination;
generating, by inputting the interaction risk vector and a time-series constraint vector received from the client terminal into a reinforcement learning model, an output vector representing an optimized allocation of time slots that minimizes a cumulative risk value derived from the interaction risk vector; and
transmitting, via the packet-switched network to the client terminal, a response data packet comprising the output vector.
